(19) United States
(12) Patent Application Publication
(10) Pub. No.: US 2023/0352132 A1
(43) Pub. Date: Nov. 2, 2023

(54) SYSTEMS AND METHODS FOR USING TEMPORAL OBJECTS FOR NATURAL LANGUAGE PROCESSING

(71) Applicant: Intelligent Medical Objects, Inc., Rosemont, IL (US)

(72) Inventors: Jonathan Gold, Louisville, CO (US); Emma Lee Foley-Beaver, Chicago, IL (US); Steven Rube, Lake Forest, IL (US); John Tian, Gurnee, IL (US); Marian Cardwell, Rosemont, IL (US); John A. Stevens, Rosemont, IL (US)

(21) Appl. No.: 17/730,790

(22) Filed: Apr. 27, 2022

(51) Int. Cl. G16H 15/00; G06F 40/211; G16H 10/60

(52) U.S. Cl. CPC G16H 15/00; G06F 40/211; G16H 10/60

(57) ABSTRACT

Systems and methods for using temporal objects for natural language processing. One system includes an electronic processor configured to receive a set of electronic records of a patient, where each electronic record is associated with an event of the patent. The electronic processor is also configured to determine a temporal statement and an associated element, where the temporal statement and the associated element are associated with the event. The electronic processor is also configured to determine a temporal characteristic for the event based on the temporal statement and the associated element. The electronic processor is also configured to generate, based on the temporal characteristic, a temporal event entry associated with the event for a profile of the patient and enable access to the temporal event entry.

### FIELD

Embodiments described herein relate to temporal objects for natural language processing, and, more particularly, to a temporal domain for the incorporation of temporality into natural language processing, data analytics, and predictive modeling.

### BACKGROUND OF THE INVENTION

Precision medicine, artificial intelligence, machine learning, data analytics, and predictive modeling hold great promise to advance healthcare-possibly as dramatically as the introduction of scientific research methodology to medicine in the past century. While the ‘big data’ healthcare analytics field swells, temporal associations or relationships is an indispensable and absent element for analytics and natural language processing (NLP) vendors, heavy data consumers, administrators, regulatory and quality assurance directors, medical and pharma researchers, public health investigators, clinical end users, and the like.

The following example illustrates the importance of temporality for robust clinical content (e.g., a robust medical profile of a patient). Consider problem lists for Patient A and Patient B. Patient A’s problem list includes diabetes, smoking history of 30 packs a year, lung cancer, and status post myocardial infarction. Similarly, Patient B’s problem list includes diabetes, smoking history of 30 packs a year, lung cancer, and status post myocardial infarction. In many cases, a lack of or limited access to temporal data impairs the general understanding of a situation (e.g., a health profile of a patient). The sequence and length of events for these patients matter (e.g., event sequencing and temporality). As one example, whether the patient smoked for 30 years prior to developing lung cancer impacts the general understanding of that patient’s health situation. As another example, whether the patient never smoked until after receiving the diagnosis of lung cancer and has smoked for 30 years since that diagnosis impacts the general understanding of that patients’ health situation.

Although traditional NLP techniques may determine and extract terms from blocks of text, traditional NLP techniques are not designed or well-suited to determine how concepts relate to one another temporally. Following the above example, while traditional NLP techniques could extract the terms, such as, e.g., “diabetes,” “smoking history of 30 packs a year,” “lung cancer,” and “status post myocardial infarction,” traditional NLP techniques are unable to assess or determine a temporal relationship between the extracted terms, let alone provide temporal insight that impacts the general understanding of a patient’s health situation.

Accordingly, there is a need for the development of temporal objects as a domain, syntactic rules, and an approach to semantic validation that provides a missing, mission critical component to support these fields. As one example, there is a need to deliver supporting domains for building profiles to enable comprehensive data analytics and predictive modeling.

### SUMMARY OF THE INVENTION

Accordingly, the present disclosure provides systems and methods that overcome one or more of the aforementioned drawbacks by providing new systems and methods for the development temporal objects for natural language processing, and, more particularly, to a temporal domain for the incorporation of temporality into natural language processing, data analytics, and predictive modeling. The embodiments described herein provide a temporal domain for building robust profiles that enable comprehensive data analytics and predictive modeling through the development of temporal objects as a domain, syntactic rules, and an approach to semantic validation.

As noted above, traditional NLP techniques are not designed or well-suited to determine how concepts relate to one another temporally. Accordingly, embodiments described herein incorporate temporality (e.g., as a temporal domain or temporal objects) into NLP techniques such that comprehensive data analytics, predictive modeling, and the like may be enhanced and improved (e.g., through the consideration of temporality or temporal relationships when performing comprehensive data analytics, predictive modeling, and the like). As one example, terms are not just extracted from a source, but temporal relationships between the extracted terms are also determined such that a robust health profile of a patient may be built and analyzed that includes or enables temporality considerations.

For example, embodiments described herein associate mathematical formulae with many common temporal phrases, which take into context when an event occurred by including the metadata of when an entry was recorded (or the patient age) and the time measurement used to describe the interval (days, weeks, months, etc.). Additionally or in addition, embodiments described herein include not only a specific point in time that the text points us to, but the likely range of time for when an event may have occurred. For a temporal phrase to be understood it will often include a specific point or range in time, a general chronology or sequence of events, or the possibility of when an event may have occurred. Plotting events on a patient’s health timeline involves some sort of measurable timeframes. With the goal of compiling a unified timeline of health-related events for a patient, organizing, and incorporating the free text found in a patient’s multiple records provides a robust reservoir of data. Accordingly, to be utilizable, temporal text must permit quantified interpretation leading to a specific point or range in time either by calling out a specific timeframe (like age or date) or giving a quantifiable time association with a timestamp and associated with either an element or event.

In accordance with one aspect of the disclosure, a system for using temporal objects for natural language processing is disclosed. The system includes an electronic processor configured to receive a set of electronic records of a patient, wherein each electronic record is associated with an event of the patent. The electronic processor is also configured to determine a temporal statement and an associated element, wherein the temporal statement and the associated element are associated with the event. The electronic processor is also configured to determine a temporal characteristic for the event based on the temporal statement and the associated element. The electronic processor is also configured to generate, based on the temporal characteristic, a temporal event entry associated with the event for a profile of the patient. The electronic processor is also configured to enable access to the temporal event entry.

In accordance with another aspect of the disclosure, a method for using temporal objects for natural language processing is disclosed. The method includes receiving, with an electronic processor, a set of electronic records of a patient, wherein each electronic record is associated with an event of the patent. The method also includes determining, with the electronic processor, a temporal statement and an associated element using at least one temporal object, wherein the temporal statement and the associated element are associated with the event. The method also includes determining, with the electronic processor, a temporal characteristic for the event based on the temporal statement and the associated element. The method also includes generating, with the electronic processor, based on the temporal characteristic, a temporal event entry associated with the event for a profile of the patient. The method also includes enabling, with the electronic processor, access to the temporal event entry.

The foregoing and other aspects and advantages will appear from the following description. In the description, reference is made to the accompanying drawings which form a part hereof, and in which there is shown by way of illustration configurations of the invention. Any such configuration does not necessarily represent the full scope of the invention, however, and reference is made therefore to the claims and herein for interpreting the scope of the invention.

### BRIEF DESCRIPTION OF THE DRAWINGS

FIG. 1 schematically illustrates components of an event and associated temporal objects according to some embodiments.

FIG. 2 schematically illustrates a system for using temporal objects for natural language processing according to some embodiments.

FIG. 3 schematically illustrates a server included in the system of FIG. 2 according to some embodiments.

FIG. 4A schematically illustrates an example high level workflow associated with the system of FIG. 2 according to some embodiments.

FIG. 4B schematically illustrates a pre-packaging stage included in the workflow of FIG. 4A according to some embodiments.

FIG. 4C schematically illustrates an importation stage included in the workflow of FIG. 4A according to some embodiments.

FIG. 4D schematically illustrates a curation stage included in the workflow of FIG. 4A according to some embodiments.

FIG. 4E schematically illustrates an evaluation stage included in the workflow of FIG. 4A according to some embodiments.

FIG. 5 is a flowchart illustrating a method for using temporal objects for natural language processing using the system of FIG. 2 according to some embodiments.

FIG. 6 is a flowchart illustrating a method for determining a temporal characteristic according to some embodiments.

FIG. 7 schematically illustrates a date generator according to some embodiments.

FIG. 8 illustrates an example patient health timeline according to some embodiments.

FIGS. 9A-9B illustrate example event matrices according to some embodiments.

FIG. 10 is a flowchart illustrating a method for performing predictive modeling according to some embodiments.

FIG. 11 illustrates an example precision matrix according to some embodiments.

FIG. 12 illustrates a table showing hypothetical entries associated with a one-time event for patient according to some embodiments.

FIG. 13 illustrates a table showing hypothetical entries for a patient who has a chronic disease according to some embodiments.

FIG. 14 illustrates a table showing hypothetical entries associated with a recurring event for a patient according to some embodiments.

### DETAILED DESCRIPTION

One or more embodiments are described and illustrated in the following description and accompanying drawings. Before any embodiments are explained in detail, it is to be understood the embodiments are not limited in their application to the details of construction and the arrangement of components set forth in the following description or illustrated in the following drawings. Other embodiments are possible, and embodiments described and/or illustrated here are capable of being practiced or of being carried out in various ways. Accordingly, the embodiments described herein may be modified in various ways and other embodiments may exist that are not described herein. Additionally, a component described as performing particular functionality may also perform additional functionality not described herein. For example, a device or structure that is “configured” in a certain way is configured in at least that way but may also be configured in ways that are not listed.

It should also be noted that a plurality of hardware and software-based devices, as well as a plurality of different structural components may be used to implement the invention. In addition, embodiments may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software (for example, stored on non-transitory computer-readable medium) executable by one or more processors. As such, it should be noted that a plurality of hardware and software-based devices, as well as a plurality of different structural components may be utilized to implement various embodiments. It should also be understood that although certain drawings illustrate hardware and software located within particular devices, these depictions are for illustrative purposes only. In some embodiments, the illustrated components may be combined or divided into separate software, firmware, and/or hardware. For example, instead of being located within and performed by a single electronic processor, logic and processing may be distributed among multiple electronic processors. Regardless of how they are combined or divided, hardware and software components may be located on the same computing device or may be distributed among different computing devices connected by one or more networks or other suitable communication links.

As used in the present application, “non-transitory computer-readable medium” comprises all computer-readable media but does not consist of a transitory, propagating signal. Accordingly, non-transitory computer-readable medium may include, for example, a hard disk, a CD-ROM, an optical storage device, a magnetic storage device, a ROM (Read Only Memory), a RAM (Random Access Memory), register memory, a processor cache, or any combination thereof.

In addition, the phraseology and terminology used herein is for the purpose of description and should not be regarded as limiting. For example, the use of “comprising,” “including,” “containing,” “having,” and variations thereof herein is meant to encompass the items listed thereafter and equivalents thereof as well as additional items. Additionally, the terms “connected” and “coupled” are used broadly and encompass both direct and indirect connecting and coupling, and may refer to physical or electrical connections or couplings. Furthermore, the phrase “and/or” used with two or more items is intended to cover the items individually and both items together. For example, “a and/or b” is intended to cover: a; b; and a and b.

As noted above, embodiments described herein provide systems and methods for the development of temporal objects for natural language processing, and, more particularly, to a temporal domain for the incorporation of temporality into natural language processing, data analytics, and predictive modeling. The embodiments described herein provide a temporal domain for building robust profiles that enable comprehensive data analytics and predictive modeling through the development of temporal objects as a domain, syntactic rules, and an approach to semantic validation. Accordingly, the embodiments described herein provide systems and methods that implement temporal associations or relationships such that conventional approaches to data analytics and predictive modeling are enhanced and improved.

Incorporation of temporality into analytics and modeling fills a gap in the interpretation of the data (e.g., precursors, outcomes, related events, and the like). Not only will its incorporation enable precision medicine and the type of phenotypic associations with patients currently being investigated for various initiatives, but its incorporation enables the derivation of meaningful links between medical treatment and health outcomes and for constructing advanced decision support systems.

A first example use case includes adding temporal objects into a single patient’s record for all curated events. As a second example use case includes enabling querying across all medical records in a system for temporal objects associated with elements (e.g., findings, problems, procedures, orders, observables, and the like). For precision medicine, temporal objects support natural language processing and may be used to evaluate data to build a patient’s longitudinal electronic medical record (LEMR), providing temporal relationships (e.g., age at event, length of event, sequence of events, time between events, and the like) from records across multiple sources of data. For population health research, by running queries through a system that has incorporated temporal relationships into the patient data (aligning patient records and being able to consider these relationships within large cohorts), the embodiments described herein may provide a fundamental tool for artificial intelligence systems and machine learning to elucidate context.

The embodiments described herein for incorporating temporality may support health information exchanges, accountable care organizations (ACOs), life sciences research, data warehouses, disease registries and future “wide area network” data sharing, thus enabling precision medicine, patient phenotypic matching, and population health studies. Moreover, inclusion of temporal objects into the patients’ records may drive data analytics and predictive modeling beyond inferred relationships to clear-cut associations between events.

Patient medical records are often distributed, resulting in varied and often inconsistent versions of an individual’s medical history. When all versions are interwoven, reconciliation of a ‘true and accurate’ history might prove a challenging (or near impossible) task. As data sources multiply, various issues arise such as which data sources can be trusted, who is charged with data governance, data stewardship, and data integrity, and the like. For example, when an event (e.g., a chronic illness or an important one-time event) is recorded in multiple records and referred to during different episodes of care, different degrees of temporal accuracy appear in the record.

The embodiments described herein address such concerns by determining the time relationships presented for events from different records and sources for a single patient, the relative veracity of data sources, construction of patient health timelines and event associations (e.g., through a LEMR), and by incorporating temporal context to data queries for large patient cohorts.

The embodiments are described herein in the context of the healthcare industry. However, it should be understood that the embodiments described herein may be implemented in the context of other industries. For example, beyond healthcare, temporal objects and spatial objects may be implemented in other industries or fields, such as, e.g., insurance claims, business models, scientific research, investigatory analyses, and the like, which often rely on the capture and interpretation of free text narratives for key tasks and construction of interlaced timelines.

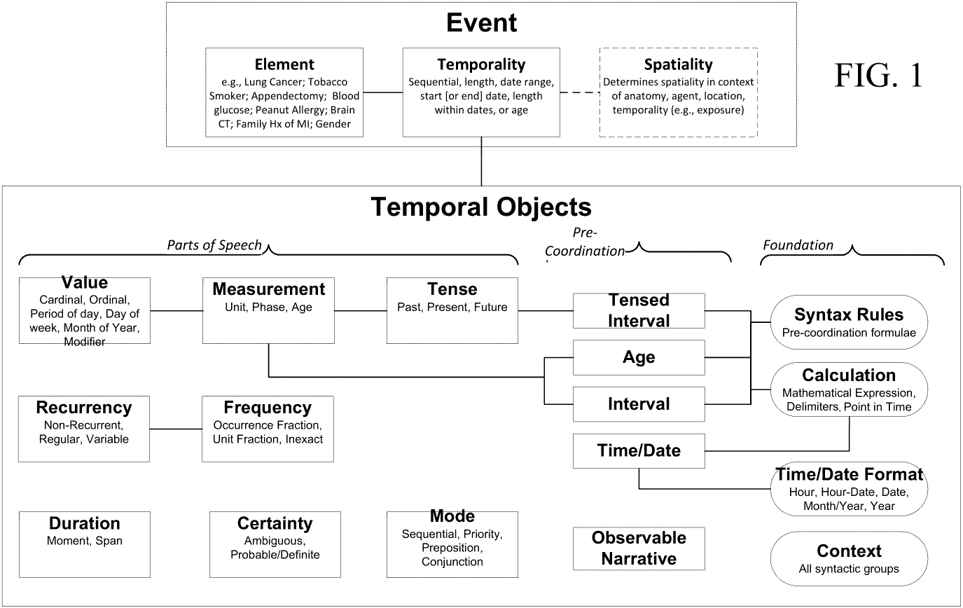

Within the context of temporality in medicine (e.g., the healthcare industry), temporality together with elements are components for events (e.g., one or more medical events), as illustrated in FIG. 1. An element may include a finding, a problem, a procedure, an order, an observable, or the like. For example, an element may be a diagnosis of lung cancer, a designation of being a tobacco smoker, an appendectomy, a blood glucose reading, a peanut allergy, a brain CT, a gender, or the like. As also illustrated in FIG. 1, an event may also be linked to spatiality (e.g., spatiality in context of anatomy, agent, location, and temporality, such as locale, exposure, etc.). As one example, when the event is a nuclear reactor leak, an element of the event may be an exposure to radiation, the temporality of the event may be 14 days prior to nuclear reactor leaking (or 14 days after leak), the spatiality of the event may be a distance from the nuclear site (e.g., 50 km, 500 km, or 5000 km). Due to its temporal qualities, an event may have an uncertain beginning or conclusion, may be ongoing, have a relationship with other events, have a sequence, may be momentary or have a span, have parts, have a cause, have a result, or have a recurrency pattern.

Temporality may describe slices of an event (or events) or the event in its entirety. Temporality may designate a period between or across events. For example, an event may occur in the past, present, future, or conditionally. Temporality may represent, e.g., a sequence, a length, a date range, a start date, an end date, a length within dates, an age, or the like. Temporal objects may be nested within additional temporal relationships. Temporal objects may be assigned an extrinsic measure (e.g., time-date) or a relation interval (e.g., age, age at occurrence, event span, time between events, or the like).

Recording events, such as in medical records, introduces metadata related to the capture of the event. Metadata related to an event may include, e.g., time/date recorded, patient ID, patient birthdate, encounter ID, facility, electronic medical record (EMR) system, document section, element domain (e.g., problem domain, procedure domain, lab result domain, medication domain, or the like), event type (e.g., recurring, non-recurring, ambiguous, one-time event, acute, chronic, or the like), author, and data source (e.g., patient, family/companion, medical report, medical claims, pharmacy, monitor, or the like).

Dates may be tethered or unlinked. A tethered date may be a derived date calculated from metadata and a relation interval (e.g., age, age at occurrence, event span, or the like). For example, a tethered date may link the date of record entry (metadata) (or a different event) to a historic, current, future, or conditional event. An unlinked date may be a specific date assigned to the event (e.g., time/date, date, month/year, year, or the like). Unless an unlinked date is fully specified (e.g., hh:mm_mm/dd/yyyy or mm/dd/yyyy), a method is used to convert the partially defined date to a specific, derived date. For example, when an unlinked date is not completely defined, the present system and method may use a derived date interpolated from the date given and the middle measure of the next closest quantifier until a fully defined month/day/year that may be used is reached. This means that the midpoint of a day (12:00pm) equals 12:00; the midpoint of a month is defined as day 15; and the midpoint of a year (day 183) equals July 2. Therefore, an unlinked event marked as occurring on 03/1995 would receive the value of Mar. 15, 1995; an unlinked event which was only listed as taking place in 2004 would be given the derived date of Jul. 2, 2004. There is an inherent rounding error using these calculations that has been deemed as acceptable. Unlinked dates for events occurring much earlier may be more reliable than tethered ones since these are given “absolute” temporal values and do not involve a calculation to determine when events took place.

Events may have different temporal perspectives. For example, an event may have a biographic perspective (e.g., the patient age when an event occurred), a differential perspective (e.g., a time measurement from one point to another point between stages in an event or between different events), and an extrinsic perspective (e.g., the time/date or date range associated with an event). A biographic perspective may be utilized when identifying patients with similar disease patterns for use in predictive modeling. A differential view may be valuable when comparing similar disease patterns, e.g., the time between the diagnosis of Diabetes Mellitus, Type 2, and the onset of chronic kidney disease. Extrinsic dates may help put a patient’s events in perspective particularly in the light of public health events (e.g., food poisoning at a restaurant, pandemic spread in a region, or the like).

As illustrated in FIG. 1, the temporality of an event may be associated with (or described by) one or more temporal objects. A temporal object may be associated with a concept (or concept grouping). In the illustrated example, a concept associated with a temporal object may be related to parts of speech, pre-coordination, calculation, and time/date format.

As illustrated in FIG. 1, concepts associated with parts of speech may include value, measurement, tense, recurrency, frequency, duration, certainty, and mode.

“Value” may represent the number, the period of the day, day of the week, month of the year, or the like. The concept of value may include, e.g., the following value categories: cardinal number (e.g., “½ of the,” “36,” “fifteen,” 27.5,” or “48-72”), ordinal number (e.g., “#7,” “third,” “secondly,” or “2^{nd}”), period of day (e.g., “during the morning,” “a.m.,” or “nighttime”), day of the week (e.g., “Sunday,” “Tues,” or “weekdays”), month of year (e.g., “April,” “Nov,” “Sep,” or “Sept”), and modifier (e.g., “4x,” “equal to,” “≥,” “lesser,” “/,” “or,” or “thru”). As one example, when a narrative provides “five days of intermittent coughing,” the term “five” is the value. As another example, when a narrative provides “every Monday, awakens with a migraine,” the phrase “every Monday” is the value. As yet another example, when a narrative provides “chills more than 3 times a week,” the phrase “more than three” is the value.

“Measurement” may serve as a type of unit associated with the value. The concept of measurement may include, e.g., the following measurement categories: unit (e.g., “hours,” “year,” “weeks-old,” “day,” or “min”) and phase (e.g., “adolescence,” “after lunch,” or “post partum”). As one example, when a narrative provides “CT scheduled four days from now,” the measurement is “days.” As another example, when a narrative provides “bleeding in first trimester,” the measurement is “trimester.”

“Tense” may designate an event as past, present, or future. Tense also allows for the extension of a past event into the present or even future, or a present event into the future. Accordingly, the concept of tense may include, e.g., the following tense categories: past (e.g., “history of,” “ago,” or “for the past”), present (e.g., “currently,” “now,” or “presently”), and future (e.g., “from now,” “scheduled,” or “shall be”). As one example, when a narrative provides “appendectomy last year,” the term “last” designates the event (i.e., appendectomy) as being a past event.

“Recurrency” or “Recurrency Pattern” may designate whether events are regularly recurrent, variably recurrent, or non-recurrent. The concept of recurrency may include, e.g., the following recurrency categories: non-recurrent (e.g., “continuously,” “single event,” or “discontinuous”), regular (e.g., “once daily,” “b.i.d.,” “qd,” or “1-2x/hr”), and variable (e.g., “periodically,” “usually,” and “multiple times”). As one example, when the narrative provides “recurrent chills, fever, malaise every three days” the term “recurrent” may indicate that the event (i.e., chills) recurs and the phrase “every three days” may indicate that recurrency pattern of the event (i.e., malaise). As another example, when the narrative provides “irregular menstruation cycles,” the term “irregular” and “cycles” may indicate a recurrency pattern of the event (i.e., menstruation).

“Frequency” may define the number of occurrences per period or units per period. The concept of frequency may include, e.g., the following frequency categories: occurrence fraction (e.g., “per 12 hours,” “/year,” and “times each hour”), unit fraction (e.g., “minutes a day,” “hr/wk,” and “hours each day”), and inexact (e.g., “occasional,” “repeated,” and “intermittent”). As one example, when a narrative provides “three times a week,” the phrase “times a week” defines a frequency (i.e., units per period) of the event. As another example, when a narrative provides “eight hours a day,” the phrase “hours a day” defines a frequency of the event.

“Duration” may relate to a moment when an event occurred or an event’s time span. The concept of duration may include, e.g., the following duration categories: moment (e.g., “acute onset” or “transient”) and span (e.g., “briefly,” “for period of,” “within,” or “lasting”). As one example, when a narrative provides “momentary lapse of consciousness,” the term “momentary” is the duration. As another example, when a narrative provides “she smoked a pack a day for twenty-five years,” the phrase “for twenty-five years” is the duration.

“Certainty” may describe the likelihood that an event occurred at a specific time or occurred at all. The concept of certainty may include, e.g., the following certainty categories: ambiguous (e.g., “possibly” or “may have had”) and probable/definite (e.g., “definitely” and “most likely occurred”). As one example, when a narrative provides “I’m pretty sure my heart attack happened in 1989,” the phrase “pretty sure” may describe a certainty associated with the event (i.e., heart attack). As another example, when a narrative provides “I may have had the mumps as a child,” the phrase “may have” describes a certainty associated with an event (i.e., mumps).

“Mode” may depict the stress of the time description (sequential) or an event (priority). A sequential mode may refer to a mode focused on an event’s sequential order or relative time (e.g., before, after, started, ended, or the like). A priority mode may refer to a mode focused on an event’s precedence (e.g., STAT, early, immediate, late, urgency, or the like). Alternatively or in addition, in some embodiments, mode contains prepositions and conjunctions that serve to define the context of a phrase. The concept of mode includes, e.g., the following mode categories: sequential (e.g., “prior”, “status post”, and “week before this”), priority (e.g., “ASAP”, “late”, “urgently”, and “early”), preposition (e.g., “above”, “before”, “during”, “for”, “in”, and “into”), and conjunction (e.g., “and”, “or”, and “if”).

As illustrated in FIG. 1, a temporal object may be associated with another concept or concept groupings, such as, e.g., a pre-coordinated related concept, a calculation related concept, a time/date format related concept.

With respect to pre-coordinated related concepts, a pre-coordinated phrase may combine value + measurement, value + time-date format, or another expression to simplify NLP concepts for dates, ages, time intervals (e.g., a designated period of time that contains both a value and a measurement unit), tensed intervals (e.g., an interval of time that includes a designation of past, present or future, such as “two days ago”, “in five weeks”, or the like), and observable narratives. An observable narrative may incorporate observable phrases associated with dates, ages, milestones, and times (e.g., “Gestational age” and “Date of Birth”). The concept of pre-coordination may include, e.g., the following pre-coordination categories: time/date (e.g., “12:24 AM”, “Jun. 25, 2017”, and “1957”), age (e.g., “age 2 weeks”, “eleven months old”, or “64 y.o.”), interval (e.g., “<2 years”, “60 days”, “54 years”, and “1 to 2 minutes”), tensed interval (e.g., “15 years ago”, “in six days”, and “1-2 hours from now”), observable narrative (e.g., “age at diagnosis” and “T wave duration”).

In some embodiments, when an interval or tensed interval is larger than one day, that interval or tensed interval may be associated with a point in time, a measure delimiter, a delimiter lower range, a delimiter upper range, or a combination thereof. Examples of pre-coordination may include “Loss of consciousness for 10 minutes after choking on food,” “15yo adolescent with rash from today,” “two months ago,” “Date of onset: Dec. 13, 2015.”

With respect to dates, due to the large number of dates and their links to other defining concepts (e.g., through concept-to-concept mapping, as described in greater detail below), in some embodiments, a date masking approach is used. The date masking approach may allow the interpretation of dates (e.g., day.month.year or month/day/year or year-month-day, month/year, year) and associate the correct point in time and delimiter dates based upon a set of rules. Table 1 (below) provides an example set of “Temporal Pre-Coordination: Time/Date” masking rules:

| | | |
|---|---|---|
| Pre-coordinated Concept | Derived Point in Time and Ranges | Measure Delimiters |
| Date and Time (Fully Defined) | Point in Time = Exact date [may be plotted on Health Timeline as Date only] | Upper and lower delimiters set to same date as point in time |
| | Exact times and dates may be necessary for key events like time of birth/death, stages for a procedure, etc. | |
| Date (Fully Defined) | Point in Time = Exact date | Upper and lower delimiters set to same date as point in time |
| Month/Year | Point in Time = 15^{th} day of specified month for specified year | measure delimiter month: ±15d |
| | Delimiter (Lower Range) = first day of month | |
| | Delimiter (Upper Range) = last day of month | |
| Year | Point in Time = 7/2 (July 2) of year for both non-leap years and leap years | measure delimiter year: ±182.5d |
| | Delimiter (Lower Range) = 1/1 (January 1) of year | |
| | Delimiter (Upper Range) = 12/31 December 31) of year | |
| Minutes:Hours of the Day | Point in Time = Exact time in minute intervals (no need for delimiters) | |
| | Exact times may be necessary for key events like time of birth/death, stages for a procedure, etc. | |

With respect to the calculation related concept grouping, calculation concepts provide mathematical expressions and points in time, which are not parts of speech, but rather help convert text to, e.g., points on a health timeline. The concept of calculation includes, e.g., the following calculation categories: mathematical expression (e.g., “calculations: date-stamp-of-entry - ” and “measure delimiter: 0.5d”), delimiter (e.g., “delimiter (lower range): <1.5d” and “delimiter (upper range): 3/25/2081”), and point in time (e.g., “point in time: date-stamp-of-entry + 9d”). The mathematical expression category provides formulae to be mapped in concept-to-concept associations with pre-coordinated intervals or tensed intervals. Mathematical expressions may include components for calculating when an event occurred when a phrase requires parsing (i.e., no pre-coordinated terms match the components). An example of a mathematical expression concept is “measure conversion week: x 7d,” which is concept-to-concept mapped to the Temporal Object concept “week(s).” The point in time category may be used to call out an “exact” date when an event has or will occur. The majority of these are associated with specific dates, but these also may appear as number of days (e.g., “point in time: 10d” is used to denote “10 days” in a mathematical formula). The delimiters category may designate two types of boundaries: (1) the earliest an event is likely to occur (as a “lower delimiter”), and (2) the latest an event is likely to occur (as an “upper delimiter”). Like the point in time category, the majority of these are associated with specific dates, but these also may appear as number of days. Concept-to-concept mapping connects concepts to formulae that in turn allow them to be mapped. Pre-coordinated, fully specified dates (month/day/year) may usually be plotted directly on a timeline. Less specific dates (month/day) require additional information for context. For these, the NLP application may infer the year by proximal words, which imply the tense for the phrase (e.g., compare “last 5/16 brain CT performed” with “on 5/16 he will undergo a brain CT”).

With respect to the time/date format related concept grouping, time and date formats vary as does the granularity used to capture a time or date (e.g., “January 6, 1950,” “6.1.1950,” “1950-01-06,” “Jan-1950,” and the like). Date concepts may use the format mm/dd/yyyy and date lexicals may use a variety of recognized formats but associate with a concept using the aforementioned format. The time/date format concept includes, e.g., the following time/date format categories: hour (e.g., “hh:mm (12-hour)” and “HH:MM (24-hour)”), hour-date (e.g., “hh:mm:dd/mm/yyyy”), date (e.g., “mm/dd/yyy,” “dd.mm.yyyy,” and “yyyy-mm-dd”), month/year (e.g., “mm/yyyy”), and year (e.g., “yyyy”).

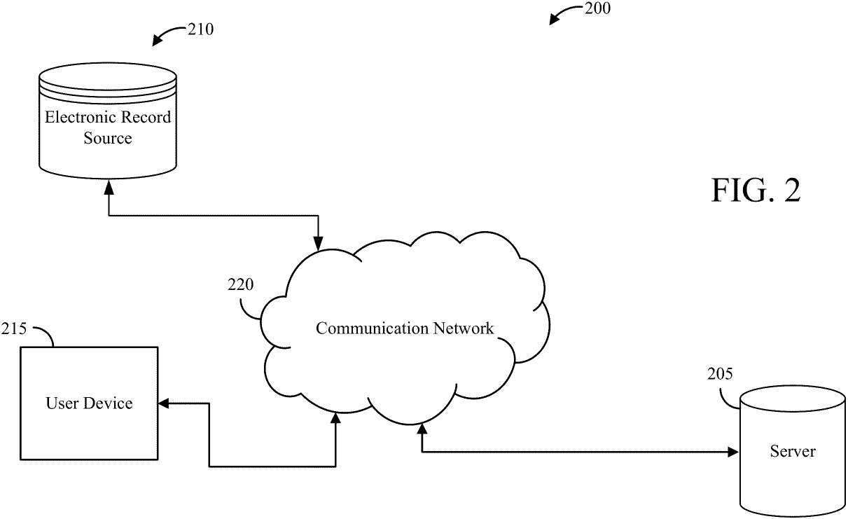

FIG. 2 illustrates a system 200 for using temporal objects for natural language processing according to some embodiments. As illustrated in FIG. 2, the system 200 includes a server 205, an electronic record source 210, and a user device 215. In some embodiments, the system 200 includes fewer, additional, or different components than illustrated in FIG. 2. For example, the system 200 may include multiple servers 205, multiple electronic record sources 210, multiple user devices 215, or a combination thereof. Also, in some embodiments, one or more components of the system 200 may be combined into a single component. As one example, the electronic record source 210 may be included in the server 205. Alternatively or in addition, in some embodiments, the functionality (or a portion thereof) described as being performed by a component of the system 200 may be distributed among multiple components.

The server 205, the electronic record source 210, and the user device 215 communicate over one or more wired or wireless communication networks 220. Portions of the communication networks 220 may be implemented using a wide area network, such as the Internet, a local area network, such as Bluetooth™ network or Wi-Fi, and combinations or derivatives thereof. It should be understood that in some embodiments, additional communication networks may be used to allow one or more components of the system 100 to communicate. Also, in some embodiments, components of the system 200 may communicate directly as compared to through a communication network 220 and, in some embodiments, the components of the system 200 may communicate through one or more intermediary devices not shown in FIG. 2.

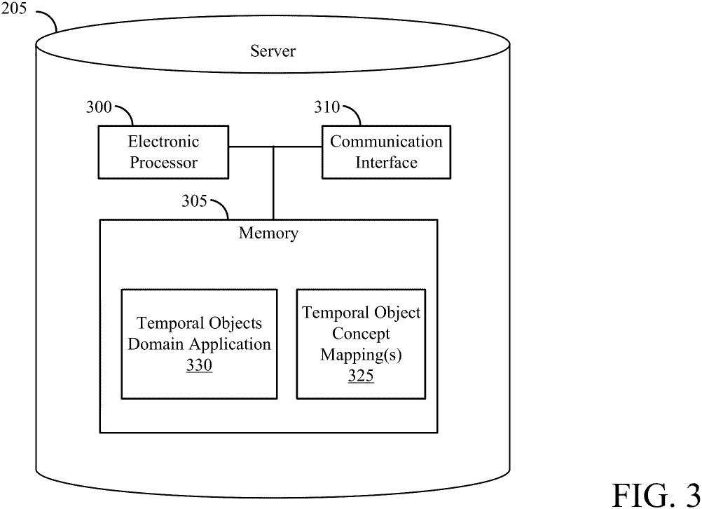

The server 205 includes a computing device, such as a server, a database, or the like. As illustrated in FIG. 3, the server 205 includes an electronic processor 300 (for example, a microprocessor, an application-specific integrated circuit (ASIC), or another suitable electronic device), a memory 305 (for example, a non-transitory, computer-readable medium), and a communication interface 310. The electronic processor 300, the memory 305, and the communication interface 310 communicate wirelessly, over one or more communication lines or buses, or a combination thereof. It should be understood that the server 205 may include additional components than those illustrated in FIG. 3 in various configurations and may perform additional functionality than the functionality described herein. For example, in some embodiments, the functionality described herein as being performed by the server 205 may be distributed among servers or devices (including as part of services offered through a cloud service), may be performed by one or more user devices 215, or a combination thereof.

The communication interface 310 allows the server 205 to communicate with devices external to the server 205. For example, as illustrated in FIG. 2, the server 205 may communicate with the electronic record source 210, the user device 215, or a combination thereof through the communication interface 310. The communication interface 310 may include a port for receiving a wired connection to an external device (for example, a universal serial bus (“USB”) cable and the like), a transceiver for establishing a wireless connection to an external device (for example, over one or more communication networks 220, such as the Internet, local area network (“LAN”), a wide area network (“WAN”), and the like), or a combination thereof.

The electronic processor 300 is configured to access and execute computer-readable instructions (“software”) stored in the memory 305. The software may include firmware, one or more applications, program data, filters, rules, one or more program modules, and other executable instructions. For example, the software may include instructions and associated data for performing a set of functions, including the methods described herein.

As illustrated in FIG. 3, the memory 305 may store a temporal object concept mapping 325. As noted above, a basic word unit in the temporal domain is the “concept” (e.g., the primary, default phrase). Precise, synonymous phrases, known as “lexicals,” serve as alternate ways for expressing the specific concept. In some embodiments, the temporal object concept mapping 325 provides a mapping of concepts to standardized medical code, such as, e.g, ICD codes, SNOMED CT concept codes, RXNorm concept codes, CPT4 concept codes, and/or other suitable standardized medical concept codes. Concepts are associated with (or mapped to) standard medical codes to the closest degree of accuracy. As one example, when a standard code may be plotted as an exact match to a concept, it is mapped as “same as.” However, when the concept is not equivalent to the full meaning of the code, but rather only part of what that code represents, it is mapped as “narrower than.” Accordingly, in some embodiments, concepts in temporal objects are mapped to SNOMED CT. Alternatively or in addition, the temporal mapping 325 provides a mapping of concepts in the temporal domain to be mapped to other concepts (e.g., utilizing concept-to-concept mapping). Accordingly, in some embodiments, concepts in the temporal domain may be mapped to other concepts. By allowing concept-to-concept mapping, the original concept may be associated with a concept that defines the term as a formula and its upper and lower limits. As one example, where the original concept is “21 days ago,” the original concept may be associated with a concept that defines the term as a formula, such as “date stamp of entry minus 21 days” and its upper and lower limits being “plus/minus one-half day.”

Temporal domain concepts provide building blocks to derive or specify as definite a timeframe as possible. As described in greater detail above, temporal objects cover many different aspects related to time-from the level of certainty to numbers to units of measurements. The approach to adding appropriate concepts is to include both clear cut temporal phrases (e.g., “January 7, 1952” and “12:53pm”), components of phrases (e.g., “minutes,” “weeks,” “times per day,” and “4”), and supporting idioms (“probably,” “currently,” and “next”).

By mapping the temporal domain concepts to a standard medical code, such as SNOMED CT, it becomes possible to group the domain concepts into temporal “parts of speech” (as described in greater detail above with respect to FIG. 1). As one example, a user may group concepts by shared SNOMED codes (utilizing the SNOMED hierarchy). Because SNOMED includes certain temporal codes and allows for certain additional components, these codes associated with concepts may be utilized to parse out phrases. This is of particular importance in natural language processing when determining whether a phrase has the correct, time-associated components to be, e.g., interpreted and plotted on a timeline.

As illustrated in FIG. 3, the memory 305 also includes a temporal objects domain application 330 (referred to herein as “the application 330”). The application 330 is a software application executable by the electronic processor 300. As described in more detail below, the electronic processor 300 executes the application 330 to develop temporal objects for natural language processing (using, e.g., the temporal object concept mapping 325), and, more particularly, to implement or provide a temporal domain for the incorporation of temporality into natural language processing, data analytics, and predictive modeling.

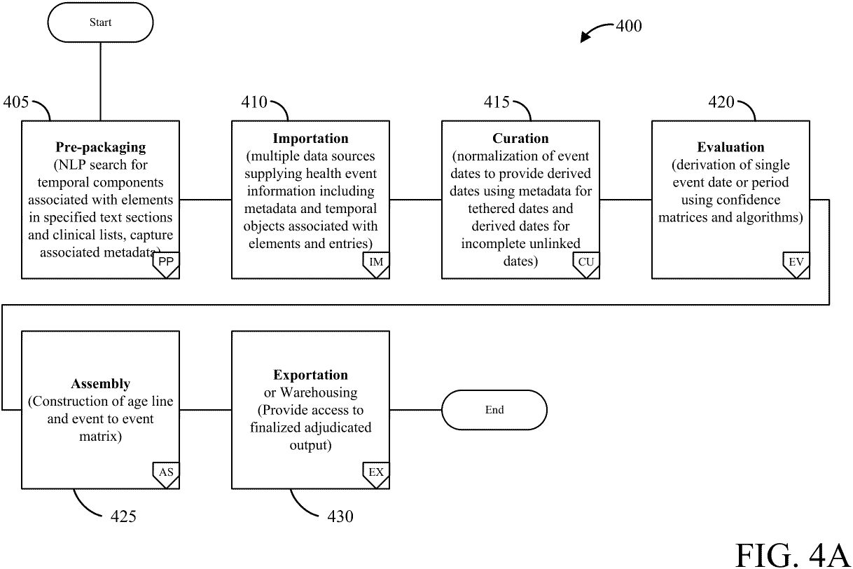

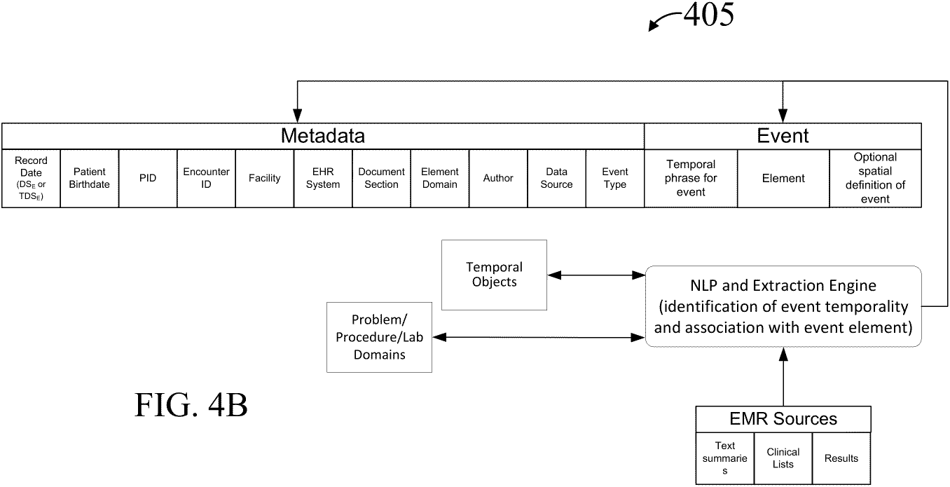

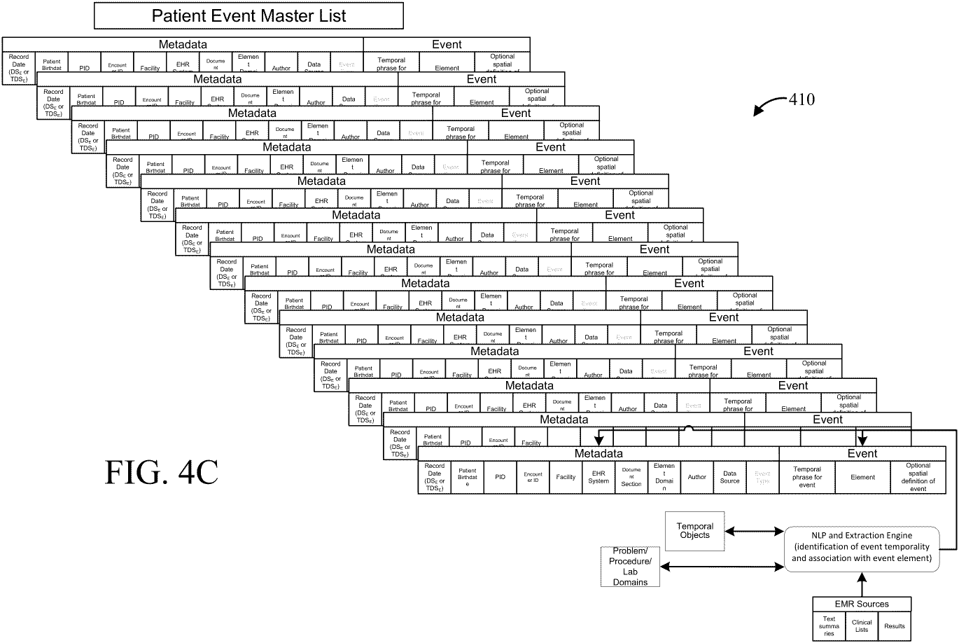

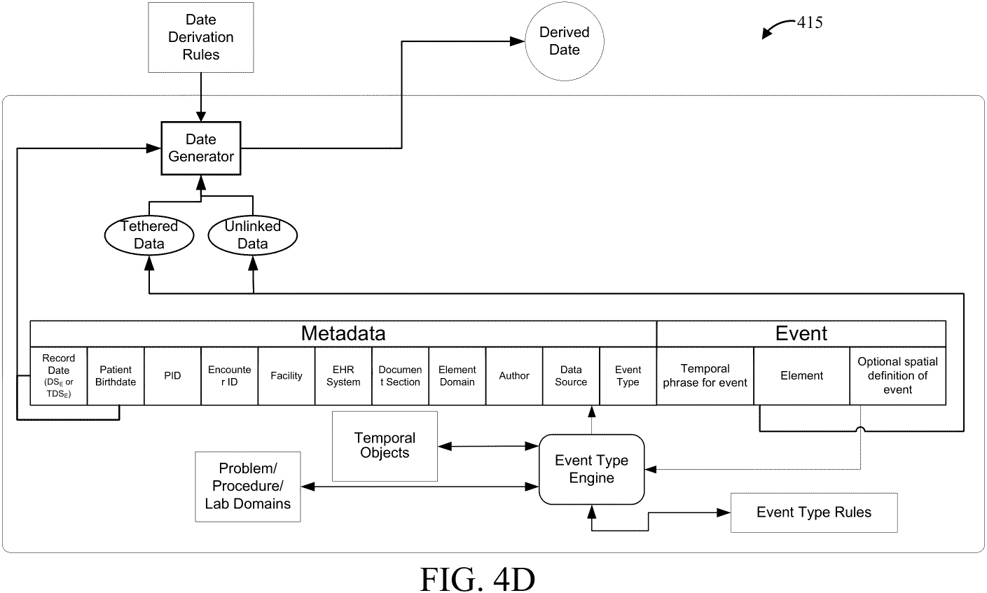

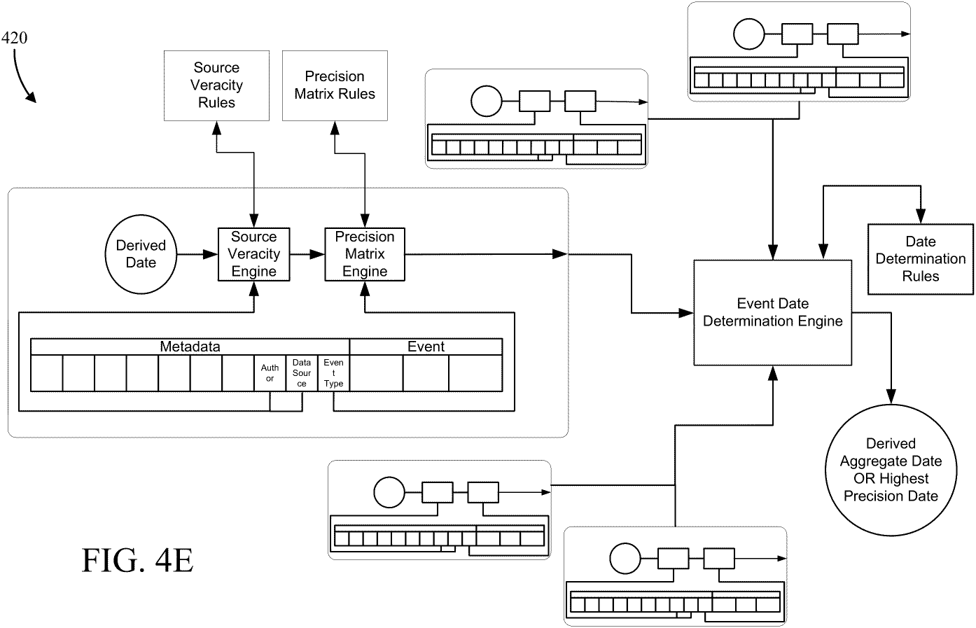

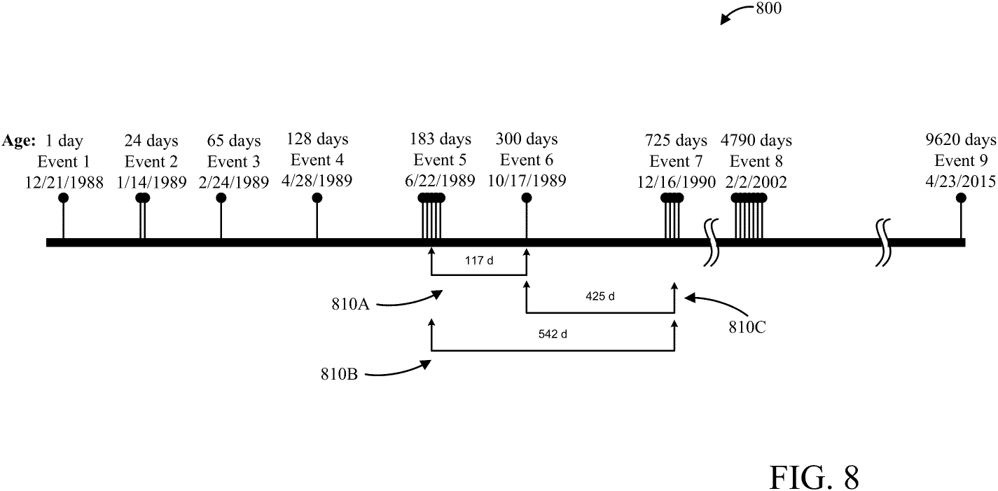

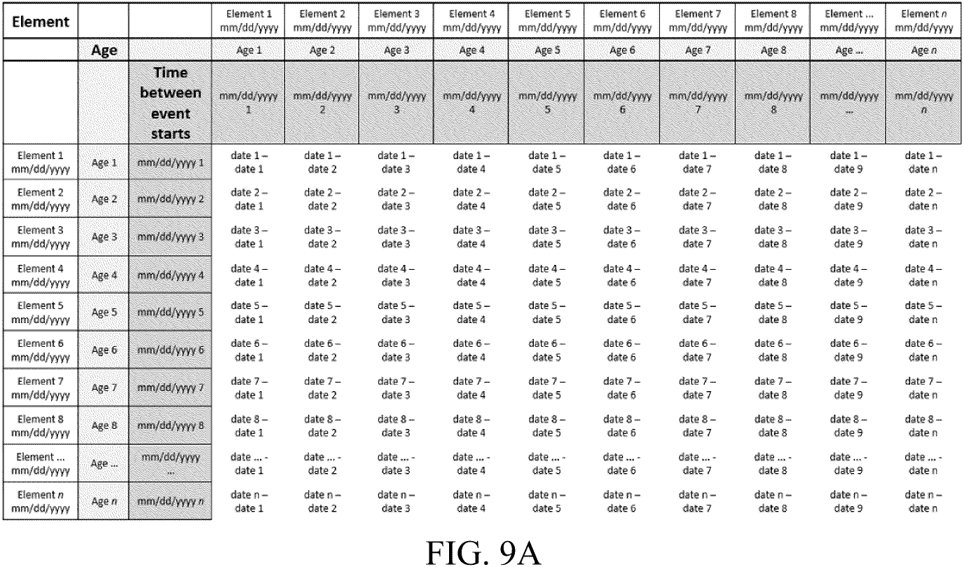

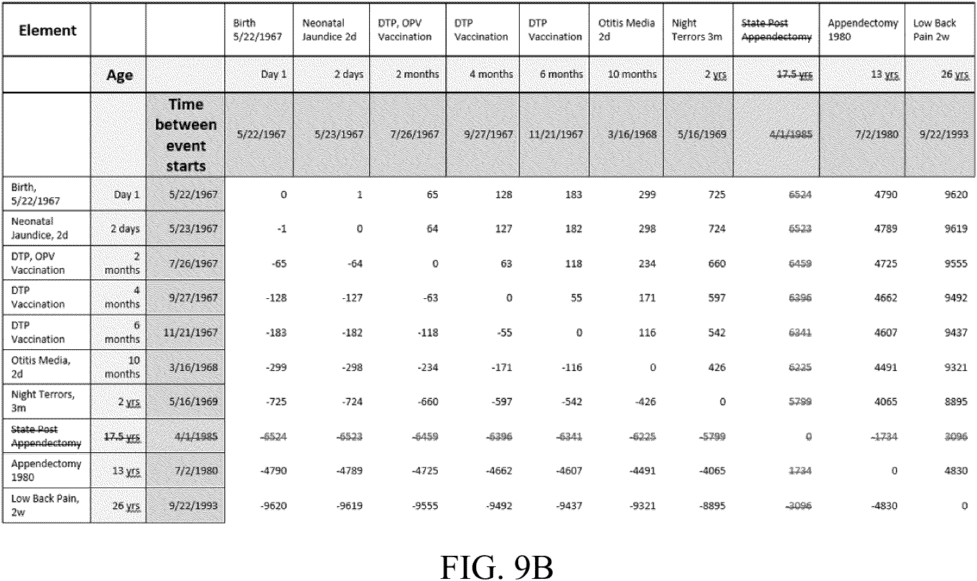

For example, FIG. 4A illustrates an example high level workflow 400 associated with functionality performed by the application 330 according to some embodiments. In the illustrated in FIG. 4A, the workflow 400 includes a pre-packaging stage 405, an importation stage 410, a curation stage 415, an evaluation stage 420, an assembly stage 425, and an exportation stage 430. The pre-packaging stage 405 includes performing a natural language processing search for temporal components associated with elements in specified text sections and clinical lists and capturing associated metadata, as illustrated in FIG. 4B. As one example, natural language processing may be used for temporal object discovery and linking raw object (e.g., “3 days ago”) to element (“fever”), associating this with encounter metadata, and adding to a patient event master list (e.g., a longitudinal medical record or health timeline). The importation stage 410 includes accessing multiple data sources supplying health event information including metadata and temporal objects associated with element and entries, as illustrated in FIG. 4C. In some embodiments, the importation stage 410 includes an import list. The import list includes each event and its related metadata for site and sent for curation, evaluation, construction and exportation back to site(s). As additional entries or records are included, these may be incorporated into final process. “Encounter ID” + “Element” may prevent an entry from being added more than once. The curation stage 415 includes performing a normalization of event dates to provide derived dates using metadata for tethered dates and derived dates for incomplete unlinked dates, as illustrated in FIG. 4D. The evaluation stage 420 includes performing a derivation of single event data or period using confidence matrices and algorithms, as illustrated in FIG. 4E. The assembly stage 425 includes constructing an age line and event to event matrix (as illustrated in FIGS. 8 and 9A-9B and described in greater detail below). The exportation stage 430 includes providing access to finalized adjudicated output. Each stage included in the example workflow 400 of FIGS. 4 will be described in greater detail below.

Returning to FIG. 2, the electronic record source 210 stores a set of or collection of electronic records, such as, e.g., electronic medical records (EMR). An electronic record may include, for example, a text summary (e.g., a summary of an appointment), clinical lists, results (e.g., a result of a procedure or test), an imaging study, and the like. The electronic records may be associated with a patient (or group of patients). For example, each electronic record may include information or data associated with an event (or medical event) associated with a patient. Metadata related to the text in which an event is captured may include, e.g., time/date recorded, patient date of birth, patient ID, encounter ID, facility, electronic medical record (EMR) system, document section, element domain (e.g., problem domain, procedure domain, lab result domain, medication domain, or the like), event type (e.g., recurring, non-recurring, ambiguous, one-time event, acute, chronic, or the like), author, data source (e.g., patient, family/companion, medical report, medical claims, pharmacy, monitor, or the like), or the like.

An electronic record source 210 may be associated with (or managed by) a record custodian or entity. As one example, the electronic record source 210 may be managed by a medical or healthcare provider organization, group, or entity. As noted above, in some embodiments, the system 100 includes multiple electronic record sources 210 (for example, a first electronic record source, a second electronic record source, a third electronic record source, and the like). In such embodiments, each electronic record source may be associated with a particular record entity (e.g., a particular medical group), a particular division of a record entity (e.g., a pharmacy of the medical group or an urgent care clinic of the medical group). As one example, a first electronic record source may be associated with a medical clinic and a second electronic record source may be associated with a pharmacy.

The user device 215 is a computing device and may include a desktop computer, a terminal, a workstation, a laptop computer, a tablet computer, a smart watch or other wearable, a smart television or whiteboard, or the like. Although not illustrated in FIG. 2, the user device 215 may include similar components as the server 205, such as electronic processor (for example, a microprocessor, an application-specific integrated circuit (ASIC), or another suitable electronic device), a memory (for example, a non-transitory, computer-readable storage medium), a communication interface, such as a transceiver, for communicating over the communication network 220 and, optionally, one or more additional communication networks or connections, and one or more human machine interfaces. For example, to communicate with the server 205, the user device 215 may store a browser application or a dedicated software application executable by an electronic processor. The system 200 is described herein as developing and implementing a temporal domain for supporting natural language processing through the server 205. However, in other embodiments, the functionality described herein as being performed by the server 205 may be locally performed by the user device 215. For example, in some embodiments, the user device 215 may store the application 330.

A user may use the user device 215 to interact with, e.g., the application 330. As one example, a user may use the user device 215 to develop or implement the temporal domain (e.g., develop temporal objects as a domain, syntactic rules, and an approach to semantic validation). Alternatively or in addition, as another example, a user may use the user device 215 to interact with the application 330 to build robust profiles (using the temporal domain), such as patient longitudinal medical record (including, e.g., a patient health timeline). Alternatively or in addition, as yet another example, a user may use the user device 215 to interact with the application 330 to perform comprehensive data analytics and predictive modeling. Accordingly, in some embodiments, a user may use the user device 215 to interact with the application 330 to perform the workflow 400 (or a portion thereof) of FIGS. 4.

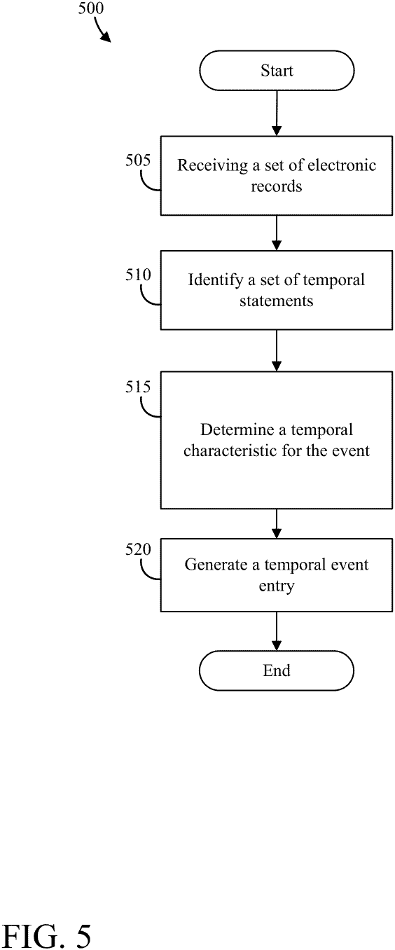

FIG. 5 is a flowchart illustrating a method 500 for using temporal objects for natural language processing performed by the system 200 according to some embodiments. The method 500 is described as being performed by the server 205 and, in particular, the application 330 as executed by the electronic processor 300. However, as noted above, the functionality described with respect to the method 500 may be performed by other devices, such as the user device 215, or distributed among a plurality of devices, such as a plurality of servers included in a cloud service.

As illustrated in FIG. 5, the method 500 includes receiving a set of electronic records (at block 505). As noted above, an electronic record may include, for example, a text summary (e.g., a summary of an appointment), clinical lists, results (e.g., a result of a procedure or test), an imaging study, and the like. The electronic record may be associated with a patient. For example, each electronic record may include information or data associated with an event (or medical event) associated with a patient. Accordingly, in some embodiments, the set of electronic records is associated with an event of a patient. As one example, the set of electronic records may describe (via, e.g., a text summary) an event of the patient, such as, e.g., a medical problem or procedure. An electronic record may include temporal data (or temporal-related data) in one or more sections of an electronic record. As one example, electronic medical record temporal sources may include a time/date stamp for entries, such as, e.g., caregiver notes, actions (e.g., medication administration, procedures, examinations, lab orders and results, imaging, etc.), routine observations (e.g., vital signs) and monitoring, free text in notes, defined time/date fields from standardized or custom forms or reports, imported data and metadata around importation, and the like.

In some embodiments, the electronic record source 210 stores a set or collection of electronic records. Accordingly, in some embodiments, the electronic processor 300 receives the set of electronic records from the electronic record source 210 via the communication network 220. Alternatively or in addition, in some embodiments, the set of electronic records may be stored in the memory 305 of the server 205. In such embodiments, the electronic processor 300 accesses (or receives) the set of electronic records from the memory 305.

Alternatively or in addition, in some embodiments, the electronic processor 300 accesses or captures metadata associated with the set of electronic records (e.g., metadata for each electronic record). As noted above, the text from each electronic record will be associated with metadata. Metadata related to text may include, e.g., time/date recorded, patient ID, encounter ID, facility, electronic medical record (EMR) system, document section, element domain (e.g., problem domain, procedure domain, lab result domain, medication domain, or the like), event type (e.g., recurring, non-recurring, ambiguous, one-time event, acute, chronic, or the like), author, data source (e.g., patient, family/companion, medical report, medical claims, pharmacy, monitor, or the like), or the like.

After receiving the set of electronic records, the electronic processor 300 determines a set of temporal statements and associated elements included in the set of electronic records (at block 510). In some embodiments, the electronic processor 300 determines a set of temporal statements using a set of syntax rules. In some embodiments, the set of syntax rules are stored in the memory 205. Alternatively or in addition, in some embodiments, the set of syntax rules are stored in a remote device or database. In such embodiments, the electronic processor 300 may access or receive the set of syntax rules through the communication network 220 from the remote device or database. Syntax rules are used to determine whether the proper parts of speech for NLP are present that will allow an event to be plotted on a timeline. In some embodiments, syntax rules are developed based on common sentence structure related to temporal statements. Initial construction of the syntax rules may include association between the most elemental and simplest phrases (e.g., a phrase using only two parts of speech, such as “last year” parsed as “Tense (Past) + Measurement (Unit year)”). Additional syntax rules may include increasing numbers of parts of speech and structures that are more complex. In some embodiments, the syntax rules for NLP are based on machine learning from electronic records and curated through clinical review.

As illustrated in FIG. 5, the electronic processor 300 may then determine a temporal characteristic for the event based on the set of temporal phrases and associated elements (at block 515). A temporal characteristic may include for example, a derived date or date range associated with the event. As one example, when the event is an appendectomy, the temporal characteristic for the appendectomy may be the date that the appendectomy was performed, as determined from the set of temporal phrases and associated elements included in electronic records associated with the appendectomy.

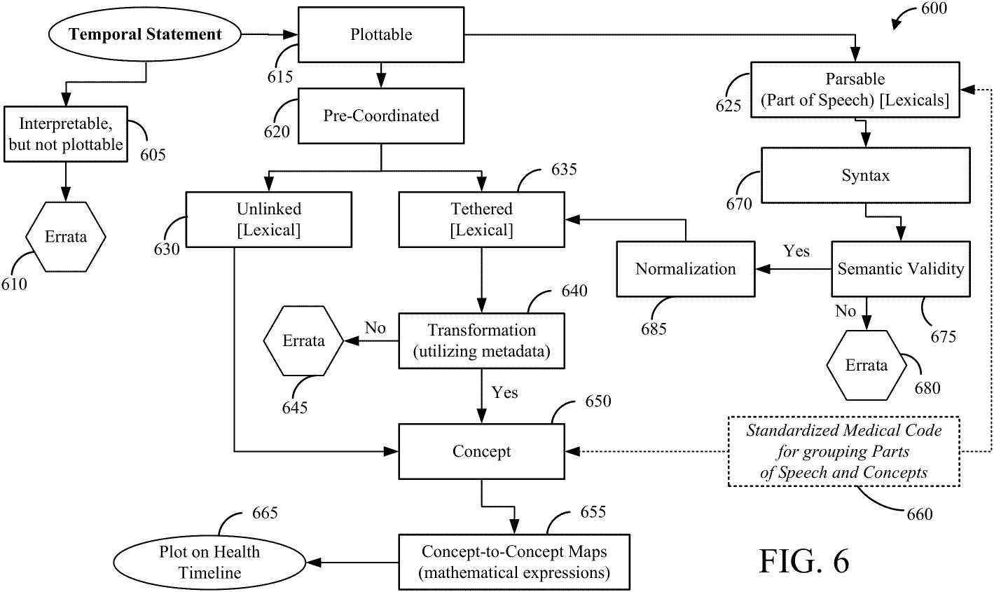

FIG. 6 is a flowchart illustrating a method 600 of determining a temporal characteristic for an event according to some embodiments. As illustrated in FIG. 6, the method 600 begins with a temporal statement (e.g., as determined by the electronic processor 300 at block 510 of FIG. 5). The electronic processor 300 determines whether the temporal statement is “interpretable” or “plottable.” An interpretable temporal statement is a temporal statement in which a temporal meaning may be inferred. An example of an interpretable temporal phrase may include: “Previously, the patient experienced headaches, but that was some time ago.” A plottable statement is a temporal statement that includes a quantifiable timeframe (e.g., the temporal statement includes the use of numbers, dates, or other clearly defined time units or phases). An example of a plottable temporal phrase may include: “Headaches beginning in May 2020.”

As illustrated in FIG. 6, when the electronic processor 300 determines that the temporal statement is interpretable, but not plottable (at block 605), the electronic processor 300 may determine that the temporal statement is errata data (at block 610). In some embodiments, in response to determining that the temporal statement is errata data, the electronic processor 300 may add the temporal statement to an errata data log. The errata data log may be stored locally, such as, e.g., in the memory 205, remotely, such as, e.g., in a remote database, or a combination thereof. An errata log lists all phrases or statements that appear to contain temporal information that cannot be plotted to a timeline (e.g., a patient’s longitudinal electronic medical record). The errata log fields may include, e.g., data origin metadata and NLP processing. Data origin metadata may include, e.g., source type, origin facility, record data, record identification, and the like. NLP processing may include, e.g., text reviewed (including +4 words pre- and post- identified words in the phrase), error message or category (e.g., syntax, semantic validity, missing metadata, missing value, ambiguous occurrence or data, duplicate or copy forward, etc.), NLP process date, NLP process facility, and the like. Alternatively or in addition, in some embodiments, the electronic processor 300 may store the errata data (i.e., the temporal statement determined to be interpretable, but not plottable) as a note to accompany a patient’s longitudinal electronic record.

When the electronic processor 300 determines that the temporal statement is plottable (at block 615), the electronic processor 300 may then determine whether the temporal statement is pre-coordinated (at block 620) or parseable (at block 625). As described in greater detail above, a pre-coordinated phrase may combine “value + measurement”, “value + measurement + tense”, “value + time-date format”, or other expressions. An example of a pre-coordinated phrase may include “May 8, 2020” or “in two weeks.” An example of a parseable temporal statement may include “every other Monday.”

When the electronic processor 300 determines that the temporal statement is a pre-coordinated phrase (at block 620), the electronic processor 300 may then determine whether the temporal statement associated with unlinked concept (at block 630) or a tethered concept (at block 635). A temporal statement that is unlinked is a temporal statement that includes a specific date assigned to an event (e.g., time/date, date, month/year, or year). Unlinked concepts may be mapped to additional concepts (e.g., concept-to-concept maps) that contain specific dates, including a point in time and upper and lower date delimiters. A temporal statement that is tethered is a temporal statement that links the date of record entry or patient’s birthdate (i.e., metadata) to a historic, current, future, or conditional event. Derived dates (e.g., temporal characteristic) may be calculated from metadata and relation interval. As one example, the temporal statement “last May” is dependent upon when the entry (i.e., the electronic record) was written (i.e., tethered to it). In this example, a date-stamp-of-entry from December 2020, would point to May 2020, whereas one from April 2020, would be associated with May 2019. Similarly, the age “74 years old” suggests that the person is that age on the day of the entry (or when an event occurred), therefore the year of birth was 74 years prior to the entry or event. Like unlinked concepts, tethered concepts utilize concept-to-concept maps. Unlike unlinked concepts, tethered concept-to-concept maps may include an intermediate step, known as “transformation,” which incorporates the metadata date-stamp-of-entry, birthdate, or referenced event date into a concept-to-concept formula to determine plottable dates (e.g., derived dates for inclusion in a patient’s longitudinal electronic health record). For example, as illustrated in FIG. 6, the electronic processor 300 may perform a transformation for temporal statements that are tethered (at block 640). The electronic processor 300 may perform a transformation by incorporating metadata into a formula to arrive at a plottable date (e.g., the addition of the date-stamp of entry to interpret the phrase “4 months ago”). The electronic processor 300 may perform a transformation using a concept-to-concept map.

As also illustrated in FIG. 6, when the electronic processor 300 cannot perform the transformation (No at block 640), the electronic processor 300 determines the temporal statement is errata data (as described in greater detail above) (at block 645). Alternatively, when the electronic processor 300 can perform the transformation (Yes at block 640), the electronic processor 300 identifies (or determines) a concept associated with the temporal statement (at block 650). As one example, where the tethered temporal statement includes “last May,” the electronic processor 300 may transform “last May” (using the date stamp of entry of Dec. 14, 2020, included in metadata) to “05/2020.” According to this example, the electronic processor 300 determines the concept associated with the temporal statement to be “05/2020.” Accordingly, the electronic processor 300 may determine or identify the concept (at block 650) based on the transformation (at block 640).

After determining the concept (at block 650), the electronic processor 300 may then perform one or more concept-to-concept mappings (at block 655). In some embodiments, the electronic processor 300 may perform the one or more concept-to-concept mappings based on the temporal object concept mappings 325 (represented in FIG. 6 by reference numeral 660). As described above, the temporal object concept mappings 325 provides a mapping of concepts to standardized medical code, such as, e.g., ICD codes, SNOMED CT concept codes, RXNorm concept codes, CPT4 concept codes, and/or other suitable standardized medical concept codes. Alternatively or in addition, the temporal mapping 325 provides a mapping of concepts in the temporal domain to be mapped to other concepts (e.g., the concept-to-concept mapping at block 655 of FIG. 6). Accordingly, in some embodiments, concepts in the temporal domain may be mapped to other concepts. By allowing concept-to-concept mapping, the original concept may be associated with a concept that defines the term as a formula and its upper and lower limits. Following the example set forth above where the tethered temporal statement includes “last May” and the concept was determined to be “05/2020,” the electronic processor 300 may determine the concept-to-concept maps to include “point in time: May 16, 2020,” “measure delimiter month: 15d,” “delimiter (lower range): May 1, 2020,” and “delimiter (upper range): May 31, 2020.”

Based on the concept-to-concept mapping (at block 655), the electronic processor 300 may determine the temporal characteristic (e.g., a derived date or date range that is plottable on a health timeline) (at block 665).

Returning to block 625 of FIG. 6, the electronic processor 300 may determine that the temporal statement does not exist as a pre-coordinated term, but is parseable. In response to determining that the temporal statement is parseable, the electronic processor 300 may parse (or deconstruct) the temporal statement. In some embodiments, the electronic processor 300 parses the temporal statement into parts of speech that are connected using rules of syntax to produce an interpretable meaning. The parts of speech are described in greater detail above. Accordingly, the electronic processor 300 may parse a temporal phrase based on syntax rules or structures (at block 670). As one example, for the temporal statement “in the past two hours,” the electronic processor 300 may parse the temporal statement as (1) (in the) + “past two hours,” (2) (in the) + “past” + “two hours,” and/or (3) (in the) + “past” + “two” + “hours.” Syntax structures for parsing the phrase may be, respectively, (1) Pre-coordinated (Tensed Interval), (2) Tense (Past) + Pre-coordinated (Interval), and (3) Tense (Past) + Value (Cardinal number) + Measurement (Unit). Each of these parsing options or techniques may be used and each should lead to the same semantic interpretation. Accordingly, rules may be used that define what component parts of speech may produce an alternative part of speech (e.g., Value (Cardinal number) + Measurement (Unit_hour) ∈ Pre-coordinated (Interval) (i.e., a number value and a measurement unit are elements of a pre-coordinated interval; Pre-coordinated (Interval) + Tense (Past) ∈ Pre-coordinated (Tensed Interval))). In some embodiments, the electronic processor 300 performs the parsing option or technique based on which parsing option is the simplest. In the example of “past two hours,” the electronic processor 300 may determine the Pre-coordinated (Tested Interval) option is the simplest. As one example, a single pre-coordinated concept may be the most basic “simplest” choice. However, when there are combinations of pre-coordinated and parsable terms, the electronic processor 300 may search for the one with the minimal number of concepts to interpret a statement. In some embodiments, the approach to natural language processing begins with an exploration for immediately interpretable pre-coordinated phrases (e.g., time/date, tensed interval, and age) followed by other pre-coordinated groups (e.g., observable narrative and interval) and then by other syntactic groups (e.g., measurement, value, tense, recurrency, frequency, duration, and mode).

If after parsing the temporal statement, the statement is found to follow syntax rules (at blocks 625 and 670), the electronic processor 300 may determine a semantic validity (at block 675). Semantic validity may depend on rules used to determine if the proper parts of speech are present and syntax correct to allow an event to be plotted on a health timeline. Semantics may refer to the meaning of a phrase. When all parts of speech in a statement obey the syntactic rules and lead to a plottable timeframe for an event, the rules may be considered semantically valid. This may result in normalization (block 685) and enable the phrase to be associated with a tethered, pre-coordinated concept (block 635). Therefore, the endpoint for using natural language when processing a temporal phrase may be to produce a specific date (e.g., an approximation of an “exact” date for an event) and a range (e.g., reasonable lower and upper limits for an event) to indicate when the event most likely occurred or will occur.

As illustrated in FIG. 6, when the electronic processor 300 determines that the semantic validity is invalid (No at block 675), the electronic processor 300 determines the temporal statement is errata data (as described in greater detail above) (at block 680). Alternatively, when the electronic processor 300 determines that the semantic validity is valid (Yes at block 675), the electronic processor 300 performs blocks 685 and 635-665, as described above.

With respect to determining an event date, temporality may either be presented as highly defined or an approximation. When an exact date (or time) is given by a trusted source (e.g., the date on a radiological study), there may be no need for including a range of when the event may have occurred. However, some sources, such as, e.g., text records, present an estimate as to when the event occurred. Accordingly, in some embodiments, to capture the timing of an event, the electronic processor 300 may determine both the specific point in time referenced by the text and a range (e.g., lower to upper limit) that may also contain the event when the source is only approximating when the event occurred. Precision varies between measurement units, such that describing an event in terms of days is a more sensitive measurement than weeks, weeks more than months, and the like.

For comparative purposes, “14 days ago” and “two weeks ago” reference the same point in time; however, when the source is approximating when the event occurred-the “rounding error” for weeks is greater than that for days. To address this potential rounding error, the electronic processor 300 may take the exact time or date deduced from the source and add a range based on a measurement unit. As one example, the electronic processor 300 may use a range that is ± ½-measurement unit (i.e., the measurement). In the above example, the range for “14 days” equals 13.5 - 14.5 days ago, whereas the range for “two weeks” equals 1½ - 2 ½ weeks (i.e., 10.5 - 17.5 days) ago. This allows for both an exact date and a range of dates to be determined using the time/date stamp on the entry.

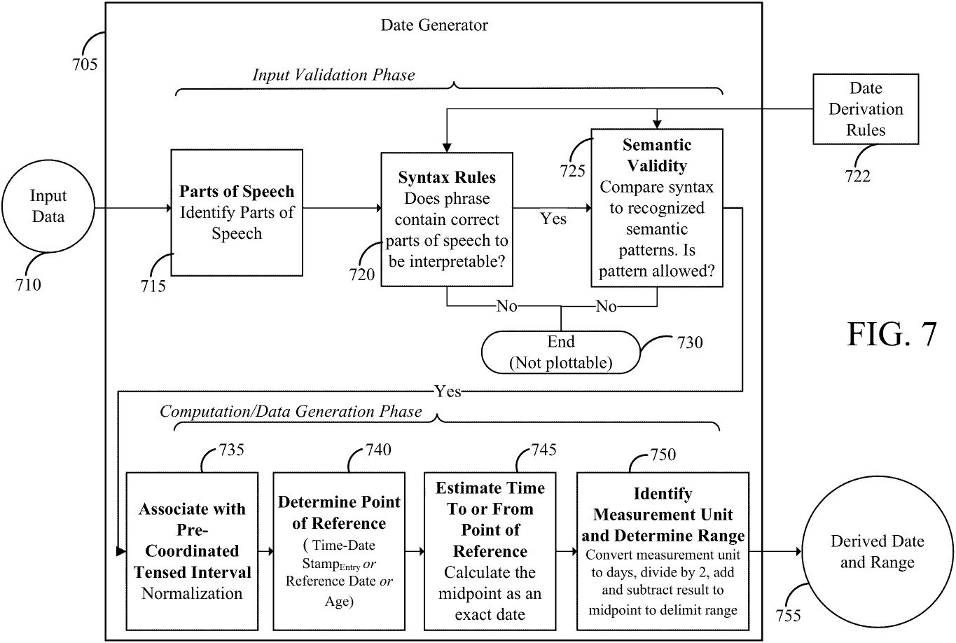

For example, FIG. 7 schematically illustrates a process for determining an event date illustrated as a date generator (e.g., software executed by the electronic processor 300, such as part of the application 330) according to some embodiments. As illustrated in FIG. 7, the date generator 705 receives input data 710. The input data 710 may include, e.g., a delimited temporal phrase, an associated element, a date-stamp-of-entry, a reference date, an age, additional metadata, tethered or unlinked, or the like. As one example, the input data 710 may be an event phrase (e.g., element + temporality, such as, “sore throat” + “beginning 3 days ago”). In response to receiving the input data 710, the date generator 705 may perform an input validation phase. As part of the input validation phase, the date generator 705 may identify parts of speech for the input data 710 (at block 715), as described in greater detail above. After identifying parts of speech for the input data 710 (at block 715), the date generator 705 may apply syntax rules (at block 720). For example, the date generator 705 may determine whether the phrase contains correct parts of speech to be interpretable. When the phrase does contain correct parts of speech to be interpretable (Yes at block 720), the date generator 705 may then perform semantic validity (at block 725). However, when the phrase does not contain correct parts of speech to be interpretable (No at block 720), the date generator 705 may determine that the phrase is not plottable (at block 730).

With respect to semantic validity (at block 725), the date generator 705 may compare syntax to recognized semantic patters to determine whether the pattern is allowed. In some embodiments, the date generator 705 may determine the semantic validity using one or more date derivation rules 722. When the pattern is not allowed (No at block 725), the date generator 705 may determine that the phrase is not plottable (at block 730). However, when the pattern is allowed (Yes at block 725), the date generator 705 may associate the input with a pre-coordinated tensed interval (block 735) which in turn enables computation/date generation phase (block 740).

As part of the computation/date generation phase, the date generator 705 determines a point of reference, such as, e.g., a time-date stamp entry, a reference date, age, or the like (at block 740). The date generator 705 may then estimate time to or from point of reference by calculating a midpoint as an exact date (at block 745) (e.g., 4 weeks ago = Time-Date Stamp of Entry minus 28 days ± ½-time unit (i.e., using this example that refers to “weeks,” DSE minus 24.5-31.5 days)). The date generator 705 may then identify the measurement unit and determine a range by, e.g., converting the measurement unit to days, dividing by two, adding and subtracting the result to midpoint to delimit range (at block 750). Based on this, the date generator 705 may output the temporal characteristic (e.g., a derived date and range). For example, the date generator 705 may provide an output of the element and date with time range in days (± ½-time unit) (e.g., sore throat start date = DSE minus 2.5-3.5 days).

For a period of time (e.g., “between 2-4 weeks ago”), the median equals 21 days, lower limit 31.5 days (i.e., 28 days [4 weeks] plus 3.5 days), upper limit equals 10.5 days (i.e., 14 days [2 weeks] minus 3.5 days). When the value is the fraction “½”, like “½ day”, “½ week”, etc., then the date generator 705 may use ± ½ of the fraction as the upper and lower bounds for the time unit (e.g., “½ year ago” = DSE minus 183 days ± 91 days [¼ year] which equals DSE minus 92-274 days). With respect to maximum values, a maximum value usually may not be prior to the patient’s date of birth. However, in some instances, some dates prior to conception and birth are important, for example, birth defects, prenatal exposures, or pregnancy-related issues (e.g., maternal risk factors like prolonged maternal exposure to a known cause of birth defects). With respect to minimal values, a minimal value may not be smaller than a value of minutes from time of entry. One exception to this may relate to ECG measurements, as these often relate to observables. In some embodiments, the date generator 705 may perform a conversion. As one example, common measurement units and physiological phases (like trimester) undergo conversion to their day equivalents when rendering a date.

Returning to FIG. 5, after determining the temporal characteristic for the event (at block 515), the electronic processor 300 generates a temporal event entry (at block 520). The temporal event entry may be associated with the event and the temporal characteristic determined for the event. In some embodiments, the temporal event entry is included in a longitudinal medical record for a patient. The longitudinal medical record for the patient may provide a robust medical profile of a patient that includes a temporal component (e.g., temporal data for each event). Accordingly, the longitudinal medical record for the patient may be made up of one or more events (e.g., one or more generated temporal event entries).

In some embodiments, the electronic processor 300 stores the temporal event entry to a medical record or profile associated with the patient (e.g., the longitudinal medical record). The electronic processor 300 may store the temporal event entry (and the longitudinal medical record) locally (e.g., in the memory 305). Alternatively or in addition, the electronic processor 300 may transmit the temporal event entry to a remote device storing the longitudinal medical record associated with the patient, such as, e.g., the user device 215, another remote device or database, or a combination thereof.

In some embodiments, the electronic processor 300 enables access to the longitudinal medical record (e.g., one or more temporal event entries included in the longitudinal medical record) such that a user may interact with the longitudinal medical record. As noted above, a user may interact with the longitudinal medical record (as a robust medical profile for the patient) in order to perform comprehensive data analytics, predictive modeling, and the like. As one example, a user may interact with the longitudinal medical record by viewing the longitudinal medical record via a display device or other human-machine interface of the user device 215.

In some embodiments, the longitudinal medical record may be displayed as a patient health timeline. For example, FIG. 8 illustrates an example patient health timeline 800 according to some embodiments. A health timeline 800 graphically displays a patient’s longitudinal medical record. As noted above, there may be three temporal perspectives for an event, biographic (e.g., the patient age when an event occurs), differential (e.g., time measurement from one point to another point between stages in an event or between different events), and extrinsic (e.g., the time/date or date range of an event). The patient health timeline 800 of FIG. 8 includes the three temporal perspectives. With respect to biographic, the patient health timeline 800 includes an age (in days) for the patient. With respect to differential, the patient health timeline 800 includes three different time measurements between events (represented in FIG. 8 by reference numerals 810A-810C). With respect to extrinsic, the patient health timeline 800 includes a date for each event.

Alternatively or in addition, in some embodiments, the patient’s longitudinal medical record may be displayed in tabular form. As one example, the patient’s longitudinal medical record may be displayed as a mileage chart (e.g., a patient’s event-to-event matrix that shows the time interval between any two events for all events). FIG. 9A illustrates an example event matrix (or mileage chart) template and FIG. 9B illustrates an example event matrix (or mileage chart) for Patient A according to some embodiments. Alternatively or in addition, in some embodiments, the patient’s longitudinal medical record may be displayed in list form, such as, e.g., a patient’s master event list that lists each event (including associated event-related data).

In some embodiments, a user may interact with the longitudinal medical record to perform predictive modeling. Current utilization of large healthcare databases focuses mainly on shared access to patient medical data, billing, and such critical strategic business concerns as data analytics, quality assurance, regulatory compliance and population health. Robust stores of medical data (e.g., patient longitudinal medical record(s)) provide for advanced clinical decision support at the point of care, real-world clinical research, and the like. Matching multiple patient characteristics enables patient-specific decision support and customized, precision medicine (e.g., medical decisions tailored to an individual). Alternatively or in addition, the systems and methods described herein enable predictive modeling by providing highly specific comparisons and guidance for similar patients through the comparison and utilization of patient longitudinal medical records (e.g., health timelines) from multiple patients.

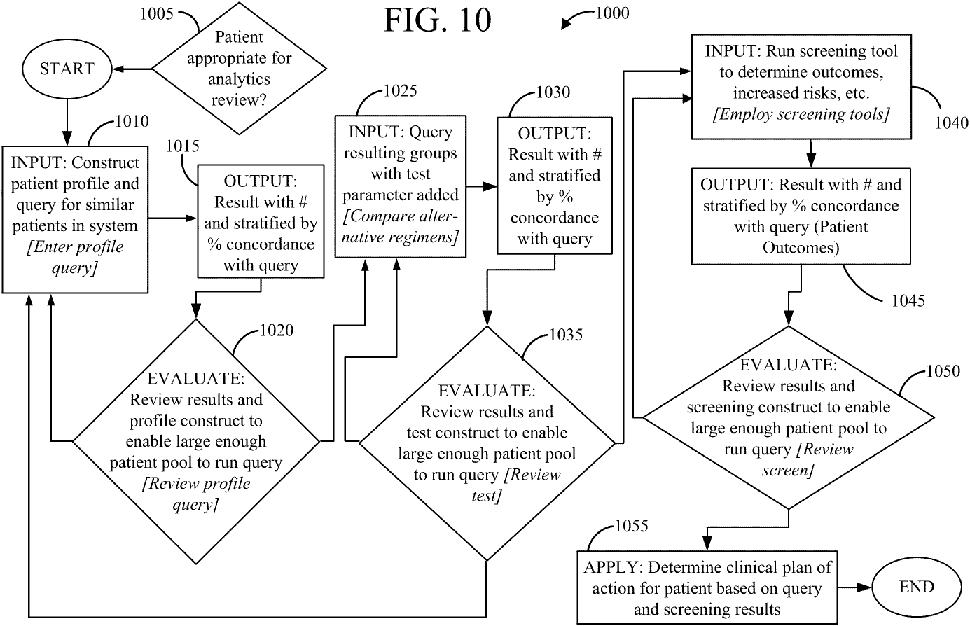

For example, FIG. 10 is a flowchart illustrating a method 1000 of predictive modeling constructed around an index patient to provide clinical guidance when determining a plan of action according to some embodiments. The method 1000 is described as being performed by the server 205 and, in particular, the application 330 as executed by the electronic processor 300. However, as noted above, the functionality described with respect to the method 1000 may be performed by other devices, such as the user device 215, or distributed among a plurality of devices, such as a plurality of servers included in a cloud service.

As illustrated in FIG. 10, the method 1000 includes an initial step of determining whether the patient is appropriate for analytics review (at block 1005). When the patient is appropriate for analytics review (Yes at block 1005), the method 1000 continues to block 1010. At block 1010, the electronic processor 300 constructs a patient profile and query for similar patients in the system (e.g., a system of a plurality of patients and associated longitudinal medical records). The electronic processor 300 may construct the patient profile as described above with respect to the method 500 of FIG. 5. The electronic processor 300 may determine a result with a number and stratified by a percentage in accordance with the query (at block 1015). The electronic processor 300 reviews results and profile construct to enable a large enough patient pool to run query (at block 1020). In some embodiments, the electronic processor 300 performs block 1020 by reviewing the profile query. The electronic processor 300 queries resulting groups with test parameter added (at block 1025) in order to determine a result with number and stratified by percentage in accordance with query (at block 1030). The electronic processor 300 reviews the results and test construct to enable a large enough patient pool to run query (at block 1035). In some embodiments, the electronic processor 300 performs block 1035 by reviewing the test. The electronic processor 300 then runs a screening tool to determine outcomes, increased risks, and the like (at block 1040). In response to employing the screening tool (at block 1040), the electronic processor 300 determines a result with a number and stratified by percentage concordance with query (e.g., patient outcomes) (at block 1045). The electronic processor 300 then reviews results and screening construct to enable large enough patient pool to run query (at block 1050). In some embodiments, the electronic processor 300 performs block 1050 by reviewing the screen. Finally, the electronic processor 300 may determine clinical plan of action for the patient based on, e.g., the query results, the screening results, or a combination thereof. In some embodiments, the clinical plan of action may be stored and/or provided to a user (via, e.g., a display device or other human-machine interface of the user device 215).

With ubiquitous electronic medical documentation and multiple provider interpretations of the patient’s history documented in numerous entries and records (e.g., multiple electronic record sources 210 of FIG. 2), conflicting accounts often arise as to when an event occurred and how long the event spanned. Accordingly, in some embodiments, the electronic processor 300 may perform an event linking process to identify when the same event is addressed in multiple records (e.g., to associate multiple versions of the same event with each other). Accordingly, in some embodiments, with respect to block 505 of FIG. 5, the electronic processor 300 may receive a plurality of electronic records. In some embodiments, one or more of the plurality of electronic records may be from different sources or from the same electronic medical source 210. Additionally, in some embodiments, when the electronic processor 300 determines that an event is associated with multiple versions, the electronic processor 300 may perform a reconciliation process. The reconciliation process may include determining what type of an event occurred (e.g., “event type”), how precise was the time or date assigned to the event (e.g., “precision”), how trustworthy was the source that reported when the event occurred (e.g., “source veracity”), and the like.

While an event may appear in only one record, often for important events, multiple entries or records from other sites may contain information or reference the same occurrence. Determining which events have multiple versions may include an identification process (e.g., an “event linking process”) followed by a reconciliation protocol or process to give the closest approximation of when an event occurred.

A combination of markers or attributes suggests that separate references (or electronic records) address the same event. The markers may be associated with a category, such as, e.g., an element category, a date category, and an event location category. The element category may include, e.g., the following markers: same element type, same IMO concept, same standardized medical code (e.g., SNOMED and/or ICD-10 or LOINC or CUI/RxNorm), same IPL cluster, reference same related labs/meds, or the like. The date category may include, e.g., the following markers: same time/date, same date, within x days, within x weeks, within x months, within one year, within x years, reference same related labs/meds, and the like. The event location category may include, e.g., the following markers: same location/site, same health system, or the like. In some embodiments, when the element is a problem, there may be a significance category and a temporal classification category of markers. The significance category may include, e.g., the following markers: near death experience (NDE), apparent life-threatening event (ALTE), organ failure, limb loss, critical condition, serious condition, and the like. The temporal classification category may include, e.g., the following markers: one-time event (e.g., an appendectomy or total abdominal hysterectomy), chronic, acute on chronic (e.g., acute exacerbation of a chronic disease), acute or finite duration event (e.g., events that are completed or that resolve within a given period, such as procedures, tests or medications), or the like. In some embodiments, the electronic processor 300 applies or assigns a score or weight to one or more markers. For example, in some instances, one marker may indicate a higher likelihood of association than another marker.

While attempting to link the same events across records, confounders make this task difficult. For instance, several discrete events may occur within a short period that may be recognized as distinct rather than a single occurrence (e.g., repeat urinalyses or recurrent ventricular arrhythmias). Accordingly, in some embodiments, the electronic processor 300 performs a categorization of temporal events (e.g., determines an event type).

In some embodiments, the electronic processor 300 may classify an event as non-recurring or recurring. Non-recurring events include one-time events (e.g., procedures that may only be performed a single time, such as an appendectomy) and the onset of most chronic disease (e.g., diabetes mellitus, type 1). Recurring events include those events that occur (or may occur) more than once (e.g., acute disease, such as an upper respiratory tract infection, medication administration, a lab test, and acute exacerbation of a chronic disease). Alternatively or in addition, in some embodiments, the electronic processor 300 may classify an event as a finite duration event or a chronic event. Finite duration events are those that are completed or that resolve within a given period. A finite duration event may include, e.g., procedures, tests, or medications. Alternatively or in addition, a finite duration event may be acute or sub-acute problems or acute exacerbations of chronic diseases. A finite duration event may be recurring (e.g., upper respiratory tract infections or blood glucose measurements) or may be non-recurring (e.g., menarche or appendectomy). While some chronic illnesses may resolve after a lengthy period (e.g., chronic otitis media), generally, chronic events do not resolve (although they may be stable or controlled). Chronic events may include illnesses, such as, e.g., hypertension, chronic kidney disease and diabetes mellitus, and may appear as open ended, dynamic, and active on a problem list. Acute exacerbations of a chronic condition (e.g., “acute exacerbation of rheumatoid arthritis”) possess dual elements-in this case, non-recurring onset of ‘rheumatoid arthritis’ and (potentially) recurring ‘acute exacerbation’. Both elements may be plotted independently on the patient’s health timeline (e.g., included as independent event entries in a patient’s longitudinal medical record) even though there may be a clear association between the two. Problems do not always align to one-time, chronic, or acute categories. As one example, atrial fibrillation may occur as an acute event or may develop into a chronic sporadic or continuous problem.

When multiple sources provide conflicting dates for the same event, the electronic processor 300 may implement additional rules related to the precision of the derived dates. The significance of the level of precision for a temporal object may become apparent when using “derived dates.” Derived dates extrapolate occurrence dates from the temporal object and the metadata (for tethered dates) or the degree of precision (for unlinked dates). All dates associated with events, whether they are fully defined and unlinked dates or derived dates, may be used to map where events should be plotted on the patient’s health timeline. By factoring in the degree of precision for each of the derived dates for a single event, the electronic processor 300 may consistently reconcile an event’s date of occurrence even when multiple sources provide conflicting dates.

The extent to which the temporal aspect of a documented event may be trusted depends upon the reliability of the temporal objects that the electronic processor 300 uses to determine the event’s date and the reliability of the source. As one example, in some instances, a one-time event may be considered the most reliable temporal object. A one-time event has a certainty which is “definite,” a value modifier of “equal,” and a value date of (hh:mm_mm/dd/yyyy), and, therefore, the date is unlinked (e.g., time of death). As another example, in some instances, a potentially recurring event may be considered the least dependable temporal object. A potentially recurring event has a certainty of ambiguous and null values for value and measure, and, therefore, the date is unlinked (e.g., previous suspected allergic reaction to bee venom). Tethered dates may be more or less specific than unlinked, historic dates.

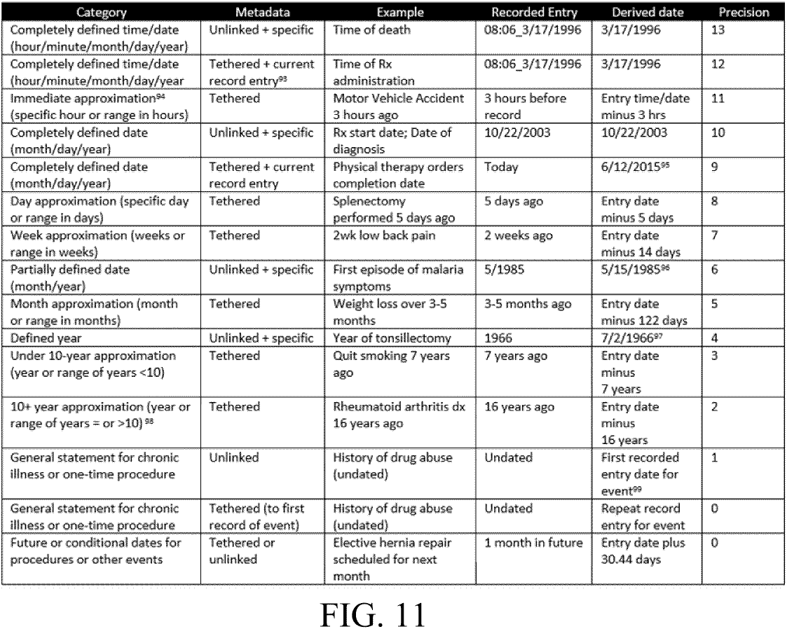

In some embodiments, the electronic processor 300 determines precision using a precision matrix (e.g., by generating or constructing a precision matrix). FIG. 11 illustrates an example precision matrix according to some embodiments. The electronic processor 300 may construct the precision matrix using a set of precision matrix rules. As one example, a precision matrix rule may provide that fully defined time/dates (hh:mm_mm/dd/yyyy) or dates (mm/dd/yyyy) are the most accurately recorded temporal points. As another example, a precision matrix rule may provide that tethered dates used for events occurring days prior to the metadata date for the medical record are more accurate than those that occur weeks prior to the record, where weeks prior to the record are more accurate than months, which in turn are more accurate than single digit years, which in turn are more accurate than double-digit years. As yet another example, a precision matrix rule may provide that tethered dates, capturing events that occurred days to weeks before an event, are more accurate than unlinked partially defined dates (month/year) and tethered dates for events occurring months prior to the medical record metadata date are more accurate than unlinked defined year dates. As yet another example, a precision matrix rule may provide that when determining an event’s start date, consider the event with the highest precision score as the correct date (e.g., the highest precision date). For any given facility, the most precise date should be considered the only event date for that database, and then the most precise representatives from all facilities (and databases) should be compared. In instances where multiple accounts of an event with different derived dates have the same high degree of precision, these should either be averaged to find a single date (mean) or, alternatively, the overlapped date(s) in ranges for the most frequent dates found should be chosen (median). As yet another example, a precision matrix rule may provide that when determining an event’s start date, implement a derived aggregate date method that considers conflicting accounts of the start date and use a hypothetical mean for the date or duration of occurrence to provide a near approximation for the actual event’s date. Each account may be weighted by its precision and source veracity before interpolating the aggregate date. This approximation is plotted on the patient’s health timeline.

In some embodiments, the electronic processor 300 determines, as part of a reconciliation process, how trustworthy a source is that reported when an event occurred (e.g., determines source veracity). The electronic processor 300 may determine source veracity as a score. In some embodiments, the electronic processor 300 determines the source veracity score based on data provenance. Data provenance may confirm the authenticity of data to enable trust in its origin and use. Provenance provides a trail accounting for the origin of a piece of data and tracking how it got to its current place in the record. Alternatively or in addition, in some embodiments, the electronic processor 300 determines the source veracity score based on an input source. For example, input sources may vary and may have different origins, such as dates entered by the patient when filling out a form or in a personal health record, time periods captured by the clinician when interviewing the patient or reviewing external consultation notes, and system generated time-dates for admission/discharge or lab reports. Depending on the type of record, dates may be attached to elements automatically (e.g., for lab results, admission time, time-date stamp of note or order entry), entered manually (e.g., by a physician assigning start dates for diagnoses on a problem list or past medical history, or by capturing events in free-text in the note section), or a combination thereof.

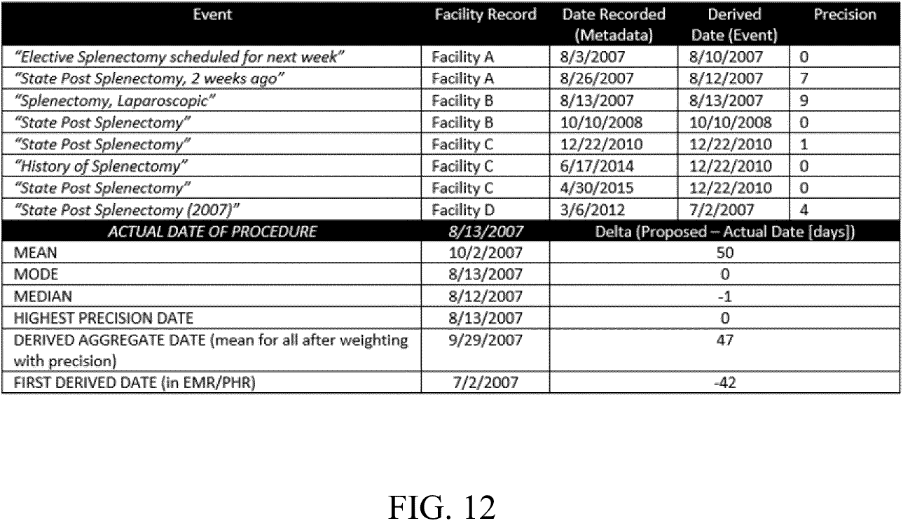

As one example of a one-time event, FIG. 12 illustrates a table showing hypothetical entries for a patient who has undergone an elective total splenectomy on Aug. 13, 2007. Facility A is the office of a surgical group practice that performs the pre-op and after care. Facility B is the local hospital where the procedure is performed. Facilities C and D are specialty clinics (endocrinology and cardiology, respectively) which see the patient years after the procedure, in 2010 and 2012, respectively. Following this example, the derived date is weighted by the precision of the temporal object. The mean of these weighted dates yields a derived aggregate date (e.g., an interpolated date for event based on derived dates and the precision for each date). The highest precision date or derived aggregate date may be plotted on the patient’s health timeline to determine patient age at event. Additional record sources, including the PHR, may shift the highest precision date and the derived aggregate date.

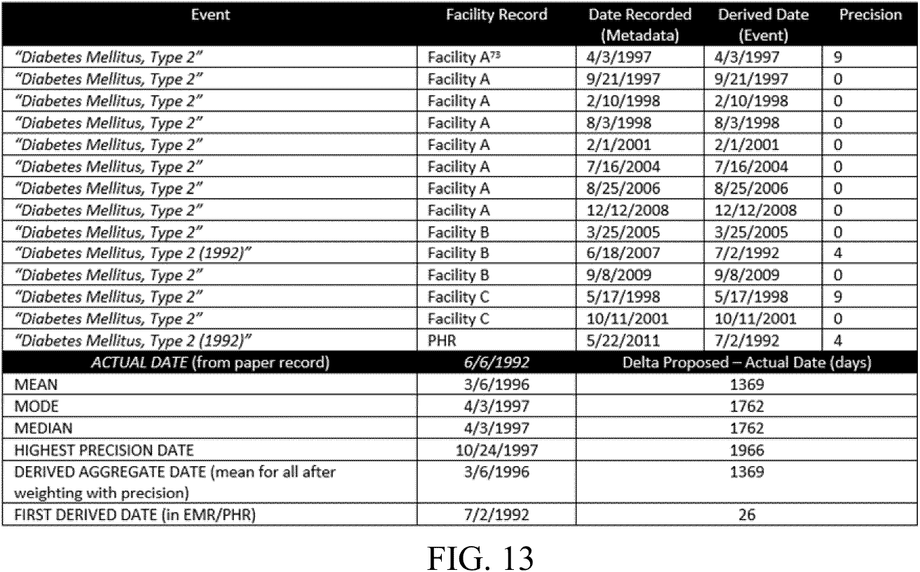

As one example of a chronic event, FIG. 13 illustrates a table showing hypothetical entries for a patient who has a chronic disease. This example illustrates how a chronic illness might be captured and how the determination of its onset may be established. Chronic illness, e.g., Diabetes Mellitus, Type 2, is a continuous condition after the initial diagnosis, hence a different strategy might be considered than that used for one-time events. Because a chronic event will typically be recorded as current, most dates will be associated with higher levels of precision (e.g., 9 or 12). An unlinked historical date (e.g., precision of 10 or 13) may provide a more accurate estimation of a chronic disease’s initial diagnosis (e.g., when historical information from a paper record is incorporated into an electronic medical record). An alternative for determining the first record for a chronic disease is to use the earliest recorded date, no matter what the precision is associated with the various later diagnosis entries. In the absence of an unlinked, historical date, the best estimate for the onset of Diabetes Mellitus, Type 2, for this patient may be the earliest record of it, since the medical record date equals the derived date and the chronic disease is current (e.g., for Facility A, this is Apr. 3, 1997; for Facility C, this is May 17, 1998). Upon inclusion of an unlinked historical date (which is prior to the first recorded disease entry), the date of diagnosis may be corrected. However, when the historical date is partially defined or has no greater precision than year and a different record from the same year shows the chronic disease as current, the derived date may be later than the first recorded date of the disease.

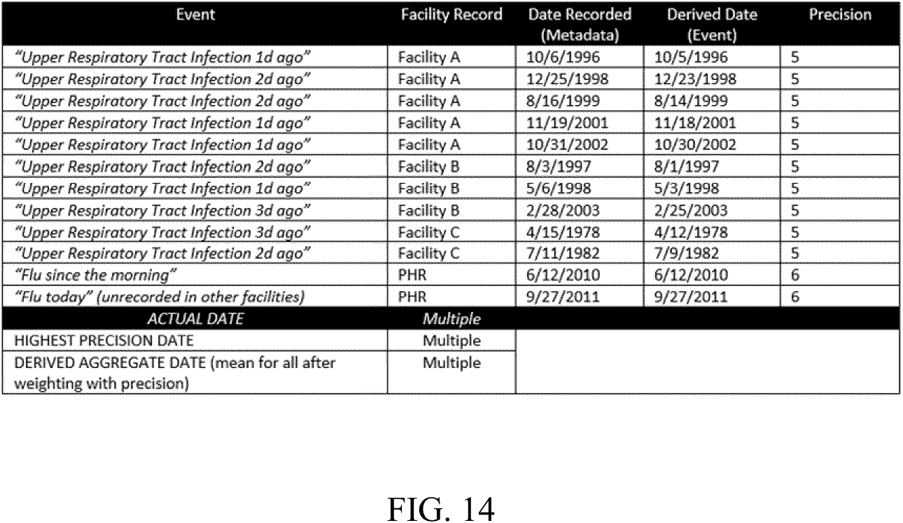

As one example of a recurring event, such as an acute disease with multiple occurrences, FIG. 14 illustrates a table showing hypothetical entries for a patient who has multiple discrete episodes of an upper respiratory tract infection at multiple different times. An acute disease differs in that it is not necessarily a one-time event, nor is it a continuous, chronic one. Unlike one-time events (where the focus may be on a short span in time) and chronic disease (where the focus is on identifying the start of the disease), acute illnesses typically are distinct, short events. Acute illnesses have beginnings and ends. Acute disease is usually recorded while the disease is active, but often the end of the disease is not documented. The beginning of the illness may be approximated anywhere from days to months prior to the diagnosis and that may be included in the record.

When events may be classified into one-time, chronic, acute, and ambiguous categories, the electronic processor 300 may use category-specific precision hierarchies or strategies to determine the date of occurrence (e.g., the temporal characteristic or derived date or range). For one-time events, the electronic processor 300 may determine (or associate) unlinked dates specific to a degree of HH:MM_mm/dd/yyyy and mm/dd/yyyy with the highest precision, followed by tethered dates to current record entry and near (hours) and close (days, weeks) approximations. Unlinked partially defined dates (mm/yyyy) may be given precedence to a tethered approximate date (months). An unlinked and defined year (yyyy) may be higher than a tethered distant (years) approximation or unlinked “occurred” record. The highest precision date may be the best option. In some embodiments, the electronic processor 300 may use a precision matrix for chronic disease. Alternatively or in addition, for chronic disease, the electronic processor 300 may determine that the first derived date (e.g., date for event based on first date cited using all tethered or unlinked results) is a more consistent option than, e.g., a precision matrix. For acute disease, the electronic processor 300 may use a precision matrix for one-time events. For ambiguous disease (e.g., “possibly had chicken pox as a child”), the electronic processor 300 may determine that temporality is not plottable. However, in some embodiments, the electronic processor 300 may include such instances (e.g., an ambiguous disease) in a listing of events deemed “not plottable” but of possible clinical importance (e.g., “polio in childhood”).

The embodiments described herein have been described in terms of one or more preferred configurations, and it should be appreciated that many equivalents, alternatives, variations, and modifications, aside from those expressly stated, are possible and within the scope of the invention.

1. A system for using temporal objects for natural language processing, the system comprising:
an electronic processor configured to
receive a set of electronic records of a patient, wherein each electronic record is associated with an event of the patent,
determine a temporal statement and an associated element, wherein the temporal statement and the associated element are associated with the event,
determine a temporal characteristic for the event based on the temporal statement and the associated element,
generate, based on the temporal characteristic, a temporal event entry associated with the event for a profile of the patient, and
enable access to the temporal event entry.

2. The system of claim 1, wherein the set of electronic records includes a first subset of electronic records and a second subset of electronic records, wherein the first subset of electronic records is received from a first electronic record source and the second subset of electronic records is received from a second electronic record source different from the first electronic record source.

3. The system of claim 1, wherein the electronic processor is configured to determine the temporal statement using natural language processing and a set of syntax rules.

4. The system of claim 1, wherein the set of syntax rules are developed based on sentence structure related to temporal statements.

5. The system of claim 1, wherein a temporal characteristic is a date associated with the event.

6. The system of claim 5, wherein the date is an approximated date in which the event occurred.

7. The system of claim 5, wherein the date is an exact date in which the event occurred.

8. The system of claim 1, wherein the electronic processor is configured to generate a health timeline for the patient, wherein the health timeline graphically represents the event chronologically along the health timeline.

9. The system of claim 1, wherein the electronic processor is configured to generate a patient event list, the patient event list including a temporal listing of events associated with the patient, wherein the temporal listing of events includes the event.

10. The system of claim 1, wherein the electronic processor is configured to determine the temporal statement and the associated element using a temporal object and natural language processing.

11. A method for using temporal objects for natural language processing, the method comprising:
receiving, with an electronic processor, a set of electronic records of a patient, wherein each electronic record is associated with an event of the patent;
determining, with the electronic processor, a temporal statement and an associated element using at least one temporal object, wherein the temporal statement and the associated element are associated with the event;
determining, with the electronic processor, a temporal characteristic for the event based on the temporal statement and the associated element;
generating, with the electronic processor, based on the temporal characteristic, a temporal event entry associated with the event for a profile of the patient, and
enabling, with the electronic processor, access to the temporal event entry.

12. The method of claim 11, wherein receiving the set of electronic records includes receiving a first subset of electronic records from a first electronic record source and receiving a second subset of electronic records from a second electronic record source different from the first electronic record source.

13. The method of claim 12, further comprising:
performing event linking across the first subset of electronic records and the second subset of electronic records,
wherein determining the temporal characteristic for the event includes applying a reconciliation protocol to each event instance included in the first subset of electronic records and the second subset of electronic records, wherein the temporal characteristic is determined based on the reconciliation protocol.

14. The method of claim 11, wherein determining the temporal statement includes applying natural language processing and a set of syntax rules to the set of electronic records.

15. The method of claim 11, further comprising:
developing syntax rules based on sentence structure related to temporal statements.

16. The method of claim 11, wherein determining the temporal characteristic includes determining a date associated with the event.

17. The method of claim 16, wherein determining the date associated with the event includes determining an approximated date in which the event occurred.

18. The method of claim 16, wherein determining the date associated with the event includes determining an exact date in which the event occurred.

19. The method of claim 11, further comprising:
generating a health timeline of the patient for display to a user, wherein the health timeline graphically represents the event chronologically along the health timeline.

20. The method of claim 11, further comprising:
generating a patient event list for display to a user, the patient event list including a temporal listing of events associated with the patient, wherein the temporal listing of events includes the event.
